(19) United States
(12) Patent Application Publication
(10) Pub. No.: US 2011/0318781 A1
(43) Pub. Date: Dec. 29, 2011

(54) CYCLODIPEPTIDE SYNTHETASE AND ITS USE FOR SYNTHESIS OF CYCLO(Tyr-Xaa) CYCLODIPEPTIDES

(75) Inventors: Pascal BELIN, Igny (FR); Muriel GONDRY, Limours-en-Hurepoix (FR); Robert THAI, Nozay (FR); Jean-Luc PERNODET, Cachan (FR); Roger GENET, Limours (FR)

(73) Assignees: COMMISSARIAT A L'ENERGIE ATOMIQUE, Paris (FR); CENTRE NATIONAL DE LA RECHERCHE SCIENTIFIQUE, Paris (FR); UNIVERSITE PARIS SUD 11, Orsay Cedex (FR); KYOWA HAKKO BIO CO., LTD, Tokyo (JP)

(21) Appl. No.: 13/165,219

(22) Filed: Jun. 21, 2011

Related U.S. Application Data
(62) Division of application No. 12/226,660, filed on Feb. 24, 2009, now abandoned.

(51) Int. Cl. C12P 21/00

(52) U.S. Cl. 435/68.1; 435/69.1

(57) ABSTRACT

Isolated, natural or synthetic polynucleotide and polypeptide encoded by said polynucleotide, that is involved in the synthesis of cyclodipeptides, recombinant vector comprising said polynucleotide or any substantially homologous polynucleotide, host cell modified with said polynucleotide or said recombinant vector and also methods for in vitro and in vivo synthesizing cyclodipeptides, in particular cyclo(Tyr-Xaa) cyclodipeptides, wherein Xaa is any amino acid and their derivatives and applications thereof.

The present invention relates to an isolated, natural or synthetic polynucleotide and to the polypeptide encoded by said polynucleotide, that is involved in the synthesis of cyclodipeptides, to the recombinant vector comprising said polynucleotide or any substantially homologous polynucleotide, to the host cell modified with said polynucleotide or said recombinant vector and also to methods for in vitro and in vivo synthesizing cyclodipeptides, in particular cyclo(Tyr-Xaa) cyclodipeptides, wherein Xaa is any amino acid and their derivatives.

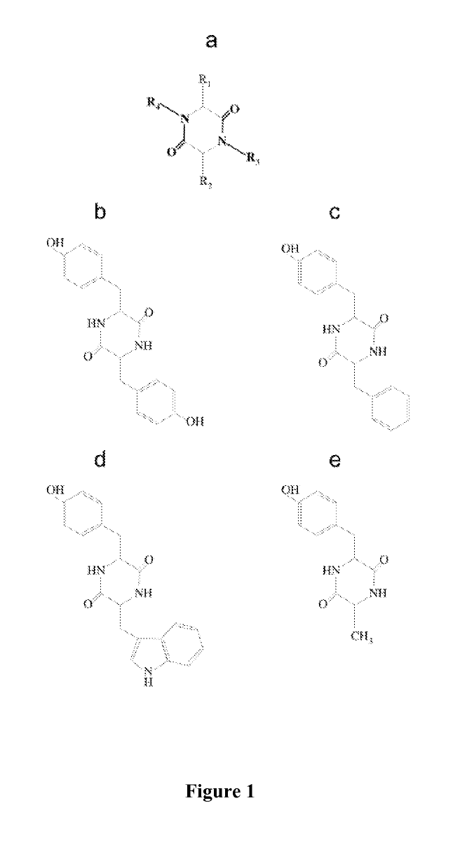

For the purposes of the present invention, the term “diketopiperazine derivatives”, “DKP”, “2,5-DKP”, “cyclic dipeptides”, cyclodipeptides or “cyclic diamino acids” is intended to mean molecules having a diketopiperazine (piperazine-2,5-dione or 2,5-dioxopiperazines) ring. In the particular case of α,β-dehydrogenated cyclodipeptide derivatives, the substituent groups R1 and R2 are α,β-unsaturated amino acyl side chains (FIG. 1). Such derivatives are hereafter referred to as “Δ” derivatives.

The DKP derivatives constitute a growing family of compounds that are naturally produced by many organisms such as bacteria, yeast, filamentous fungi and lichens. Others have also been isolated from marine organisms, such as sponges and starfish. An example of these derivatives: cyclo(L-His-L-Pro), has been shown to be present in mammals.

The DKP derivatives display a very wide diversity of structures ranging from simple cyclodipeptides to much more complex structures. The simple cyclodipeptides constitute only a small fraction of the DKP derivatives, the majority of which have more complex structures in which the main ring and/or the side chains comprise many modifications: introduction of carbon-based, hydroxyl, nitro, epoxy, acetyl or methoxy groups, and also the formation of disulfide bridges or of heterocycles. The formation of a double bond between two carbons is also quite widespread. Certain derivatives, of marine origin, incorporate halogen atoms.

Useful biological properties have already been demonstrated for some of the DKP derivatives. Bicyclomycin (Bicozamine™) is an antibacterial agent used as food additive to prevent diarrhea in calve and swine (Magyar et al., J. Biol. Chem., 1999, 274, 7316-7324). Gliotoxin has immunosuppressive properties which were evaluated for the selective ex vivo removal of immune cells responsible for tissue rejection (Waring et al., Gen. Pharmacol., 1996, 27, 1311-1316). Several compounds such as ambewelamides, verticillin and phenylahistin exhibit antitumour activities involving various mechanisms (Chu et al., J. Antibiot. (Tokyo), 1995, 48, 1440-1445; Kanoh et al., J. Antibiot. (Tokyo), 1999, 52, 134-141; Williams et al., Tetrahedron Lett., 1998, 39, 9579-9582).

Many others like albonoursin produced by Streptomyces noursei, display antimicrobial activities (Fukushima et al., J. Antibiot. (Tokyo), 1973, 26, 175-176). Cyclo(Tyr-Tyr) and cyclo(Tyr-Phe) were shown to be potential cardioactive agents: cyclo(Tyr-Tyr) being a potential cardiac stimulant and cyclo(Tyr-Phe) being a cardiac inhibitor (Kilian et al., Pharmazie 2005, 60, 305-309). These two cyclodipeptides were also tested as receptor interacting agents and the two compounds were found to exhibit significant binding to opioid receptors (Kilian et al., precited). Moreover, they were evaluated as antineoplastic agents and cyclo(Tyr-Phe) was shown to induce growth inhibition of three different cultured cell lines (Kilian et al., precited). It has been described that cyclo(ΔAla-L-Val) produced by Pseudomonas aeruginosa could be involved in interbacterial communication signals (Holden et al., Mol. Microbiol. 1999, 33, 1254-1266). Other compounds are described as being involved in the virulence of pathogenic microorganisms or else as binding to iron or as having neurobiological properties (King et al., J. Agr. Food Chem., 1992, 40, 834-837; Sammes, Fortschritte der Chemie Organischer Naturstoffe, 1975, 32, 51-118; Alvarez et al., J; Antibiot., 1994, 47, 1195-1201).

Although the number of known DKPs is increasing steadily, biosynthesis pathways of these compounds are still largely unexplored, leading to little knowledge regarding their synthesis.

In several cases reported so far, the formation of DKPs occurs spontaneously from linear dipeptides for which the cis-conformation of the peptide bond is favoured by the presence of an N-alkylated amino acid or a proline residue. Such spontaneous cyclisation has also been observed in the course of non ribosomal peptide synthesis of gramicidin S and tyrocidine A in Bacillus brevis, due to the instability of the thioester linkage during peptide elongation on peptide synthetase megacomplexes (Schwarzer et al., Chem. Biol, 2001, 8, 997-1010). Thus, in all of the known mechanisms of spontaneous DKP formation, the primary structure of the precursor dipeptide, in particular the conformation of its peptide bond, appears to be a fundamental requirement for the formation of the DKP ring to take place and for the process to result in the production of the final DKP derivative.

However, such a spontaneous cyclisation reaction cannot account for the biosynthesis of the large majority of DKP derivatives that do not contain a proline residue or an N-alkylated residue.

Known methods for producing DKP-derivatives include chemical synthesis, extraction from natural producer organisms and also enzymatic methods:
Chemical methods can be used for synthesizing DKP derivatives (Fischer, J. Pept. Sci., 2003, 9, 9-35) but they are considered to be disadvantageous in respect of cost and efficiency as they often necessitate the use of protected amino acyl precursors and lead to the loss of stereochemical integrity. Moreover, they are not environment-friendly methods as they use large amounts of organic solvents and the like.
Extraction from natural producer organisms can be used but the productivity remains low because the contents of desired DKP-derivatives in natural products are often low.
Enzymatic methods, i.e. methods utilizing enzymes either in vivo (e.g. culture of microorganisms expressing cyclodipeptide-synthesizing enzymes or microorganism cells isolated from the culture medium) or in vitro (e.g. purified cyclodipeptide-synthesizing enzymes) can be used. Enzymes known to produce cyclodipeptides are non-ribosomal peptide synthetases (hereinafter referred to as NRPS) (Gruenewald, et al., Appl. Environ. Microbiol., 2004, 70, 3282-3291) and AlbC which is a cyclodipeptide synthetase (CDS) (Lautru et al., Chem. Biol., 2002, 9, 1355-1364; International Application WO 2004/000879):
the enzymatic method utilizing NRPS has already been reported to produce a specific cyclodipeptide. The two genes coding for the bimodular complex TycA/TycB1 from Bacillus brevis (Mootz and Marahiel, J. Bacteriol., 1997, 179, 6843-6850) were coexpressed in Escherichia coli and gave rise to the production of cyclo(DPhe-Pro) (Gruenewald et al., Appl. Environ. Microbiol., 2004, 70, 3282-3291). The cyclodipeptide was stable, not toxic to E. coli and secreted in the culture medium. However, the methods utilizing NRPS appear essentially restricted to the production of cyclodipeptides containing N-alkylated residues. Moreover, the methods utilizing NRPS are difficult to implement: NRPS being large multimodular enzyme complexes, they are not easy to manipulate both at the genetic or biochemical levels.
the enzymatic method utilizing AlbC was also described to produce specific cyclodipeptides. The expression of AlbC from Streptomyces noursei by heterologous hosts Streptomyces lividans TK21 or E. coli led to the production of two cyclodipeptides, cyclo(Phe-Leu) and cyclo(Phe-Phe) that were secreted in the culture medium (Lautru et al., Chem. Biol., 2002, 9, 1355-1364). AlbC catalyzes the condensation of two amino acyl derivatives to form cyclodipeptides containing or not containing N-alkylated residues, by an unknown mechanism. This unambiguously shows that a specific enzyme unrelated to non ribosomal peptide synthetases can catalyze the formation of DKP derivatives: AlbC is the first example of an enzyme that is directly involved in the formation of the DKP motif.

Furthermore the obtained cyclo(Phe-Leu) cyclodipeptide may be transformed into a cyclo(α,β-dehydro-dipeptide), i.e. albonoursin, or cyclo(ΔPhe-ΔLeu), an antibiotic produced by Streptomyces noursei, in the presence of cyclic dipeptide oxydase (CDO) which specifically catalyzed the formation of albonoursin, in a two-step sequential reaction starting from the natural substrate cyclo(L-Phe-L-Leu) leading first to cyclo (ΔPhe-L-Leu) and finally to cyclo (ΔPhe-ΔLeu) corresponding to albonoursin (Gondry et al., Eur. J. Biochem., 2001, 268, 1712-1721). Said CDO may also transform various cyclodipeptides into α,β-dehydrodipeptides (Gondry M. et al., Eur. J. Biochem., 2001, precited).

The DKP derivatives exhibit various biological functions, making them useful entities for the discovery and development of new drugs, food additives and the like. Accordingly, it is necessary to be able to have large amounts of these compounds available.

An understanding of the pathways for the natural synthesis of the diketopiperazine derivatives could enable a reasoned genetic improvement in the producer organisms, and would open up perspectives for substituting or improving the existing processes for synthesis (via chemical or biotechnological pathways) through the optimization of production and purification yields. In addition, modification of the nature and/or of the specificity of the enzymes involved in the biosynthetic pathway for the diketopiperazine derivatives could result in the creation of novel derivatives with original molecular structures and with optimized biological properties.

The Inventors have now obtained a new cyclodipeptide synthesizing enzyme (or cyclodipeptide synthetase or CDS) family which is able to catalyze the formation of cyclo(Tyr-Xaa) cyclodipeptides, wherein Xaa is any proteinogenic or non-proteinogenic amino acid, preferably Xaa is selected among amino acid bearing an aromatic or an alkyl side chain, and even more preferably, Xaa is selected among Tyr, Phe, Trp, and Ala.

Therefore an object of the present invention is an isolated cyclodipeptide synthetase, characterized in that:
it has the ability to produce cyclo(Tyr-Xaa) cyclodipeptides from two amino acids Tyr and Xaa, wherein Xaa is any amino acid, and
it comprises a polypeptide sequence having at least 40% identity or at least 60% similarity with the polypeptide of sequence SEQ ID NO: 3.

Preferably, cyclodipeptide synthetase of the invention comprises a polypeptide sequence having at least 50% identity or at least 70% similarity, even more preferably at least 60% identity or at least 80% similarity, and even more preferably at least 70% identity or at least 90% similarity with the sequence SEQ ID NO:3.

Preferably, a cyclodipeptide synthetase of the invention comprises a polypeptide sequence having at least 75% identity or at least 95% similarity, more preferably at least 80% identity or at least 98% similarity, and most preferably at least 90% identity or at least 99% similarity, with the polypeptide of sequence SEQ ID NO:3.

According to an advantageous embodiment of the invention, said isolated cyclodipeptide synthetase of the invention is selected in the group consisting of the sequences SEQ ID NO:3, the SEQ ID NO:4 and the SEQ ID NO:6.

According to an alternative embodiment of the invention, said cyclodipeptide synthetase has a polypeptide sequence other than SEQ ID NO:4.

The percentages of identity and the percentages of similarity defined herein can be obtained using the BLAST program (blast2seq, default parameters) (Tatutsova and Madden, FEMS Microbiol Lett., 1999, 174, 247-250) over a comparison window consisting of the full-length of the sequence SEQ ID NO: 3 and preferably by calculating them on an overlap representing at least 90% of the length of SEQ ID NO:3, i.e. in 217 aminoacid overlap.

Another object of the present invention is an isolated polynucleotide selected from:

a) a polynucleotide encoding a cyclodipeptide synthetase of the present invention, as defined above;

b) a complementary polynucleotide of the polynucleotide a);

c) a polynucleotide which hybridizes to polynucleotide a) or b) under stringent conditions.

Said isolated polynucleotide advantageously hybridizes to a complementary polynucleotide of the polynucleotide sequence of SEQ ID NO:1 under stringent conditions, and encodes a cyclodipeptide synthesizing enzyme having the ability to produce cyclo(Tyr-Xaa), wherein Xaa is any amino acid.

The term “hybridize(s)” as used herein refers to a process in which polynucleotides hybridize to the recited nucleic acid sequence or parts thereof. Therefore, said nucleic acid sequence may be useful as probes in Northern or Southern Blot analysis of RNA or DNA preparations, respectively, or can be used as oligonucleotide primers in PCR analysis dependent on their respective size. Preferably, said hybridizing polynucleotides comprise at least 10, more preferably at least 15 nucleotides while a hybridizing polynucleotide of the present to be used as a probe preferably comprises at least 100, more preferably at least 200, or most preferably at least 500 nucleotides.

It is well known in the art how to perform hybridization experiments with nucleic acid molecules, i.e. the person skilled in the art knows what hybridization conditions she/he has to use in accordance with the present invention. Such hybridization conditions are referred to in standard text books such as Sambrook et al., Molecular Cloning: A Laboratory Manual; Cold Spring Harbor Laboratory Press, 2^{nd} edition 1989 and 3^{rd} edition 2001; Gerhardt et al.; Methods for General and Molecular Bacteriology; ASM Press, 1994; Lefkovits; Immunology Methods Manual: The Comprehensive Sourcebook of Techniques; Academic Press, 1997; Golemis; Protein-Protein Interactions: A Molecular Cloning Manual; Cold Spring Harbor Laboratory Press, 2002 and other standard laboratory manuals known by the person skilled in the Art or as recited above. Preferred in accordance with the present inventions are stringent hybridization conditions.

“Stringent hybridization conditions” refer, e.g. to an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulfate, and 20 μg/ml denatured, sheared salmon sperm DNA, followed e.g. by washing the filters in 0.2×SSC at about 65° C.

Also contemplated are nucleic acid molecules that hybridize at low stringency hybridization conditions. Changes in the stringency of hybridization and signal detection are primarily accomplished through the manipulation of formamide concentration; salt conditions, or temperature. For example, lower stringency conditions include an overnight incubation at 37° C. in a solution comprising 6×SSPE (20×SSPE=3 mol/l NaCl; 0.2 mol/l NaH₂PO₄; 0.02 mol/l EDTA, pH 7.4), 0.5% SDS, 30% formamide, 100 μg/ml salmon sperm blocking DNA; followed by washes at 50° C. with 1×SSPE, 0.1% SDS.

In addition, to achieve even lower stringency, washes performed following stringent hybridization can be done at higher salt concentrations (e.g. 5×SSC). It is of note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations.

An example of an isolated polynucleotide of the invention is the polynucleotide of sequence SEQ ID NO: 2 corresponding to the sequence known as Rv2275 gene, isolated from Mycobacterium tuberculosis (SEQ ID NO: 2 corresponds to positions from 2546883 to 2547752 of Mycobacterium tuberculosis H37Rv complete genome of GenBank accession number GI:57116681) and which encodes a cyclodipeptide synthetase of sequence SEQ ID NO: 4. The information available on the different databases concerns hypothetical proteins, which were not isolated and for which no function has been defined.

Another example of an isolated polynucleotide of the invention is the polynucleotide of sequence SEQ ID NO: 5 corresponding to a variant of SEQ ID NO: 2 wherein 2^{nd} codon is a GCA codon (instead of a TCA codon in SEQ ID NO:2) and which encodes a cyclodipeptide synthetase of sequence SEQ ID NO: 6 wherein 2^{nd} amino acid is Ala (instead of Ser). A further example of an isolated polynucleotide of the Invention is the polynucleotide of the sequence SEQ ID NO: 1 corresponding to the 5′-truncated sequence of SEQ ID NO: 2 starting from 49^{th} codon (TTT) wherein said 49^{th} codon is replaced with an ATG codon and 50^{th} codon is either unchanged (CAG) or replaced with a GAG codon, and which encodes a truncated form of a cyclodipeptide synthetase of sequence SEQ ID NO: 3 wherein 2^{nd} amino acid is either Gln or Glu.

According to an alternative embodiment of the invention, said polynucleotide has a polynucleotide sequence other than SEQ ID NO:2.

Isolated polynucleotides of the invention can be obtained from DNA libraries, particularly from Mycobacterium DNA libraries, for example from a Mycobacterium tuberculosis or a Mycobacterium bovis DNA library, using SEQ ID NO: 1 as a probe. Polynucleotides of the invention can also be obtained by means of a polymerase chain reaction (PCR) carried out on the total DNA of a Mycobacterium, in particular of Mycobacterium tuberculosis or Mycobacterium bovis, or can be obtained by RT-PCR carried out on the total RNA of a Mycobacterium, in particular of Mycobacterium tuberculosis or Mycobacterium bovis.

Another object of the present invention is a recombinant vector characterised in that it comprises a polynucleotide of the present invention as defined above.

The vector used may be any known vector of the prior art. As vectors that can be used according to the invention, mention may in particular be made of plasmids, cosmids, bacterial artificial chromosomes (BACs), integrative elements of actinobacteria, viruses or else bacteriophages.

Said vector may also comprise any regulatory sequences required for the replication of the vector and/or the expression of the polypeptide encoded by the polynucleotide (promoter, termination sites, etc.).

Another object of the invention is a modified host cell into which a polynucleotide or a recombinant vector of the invention as defined above, has been introduced.

Such a modified host cell may be any known heterologous expression system using prokaryotes or eukaryotes as hosts, and is preferably a prokaryotic cell. By way of example, mention may be made of animal or insect cells, and preferably of a microorganism and in particular a bacterium such as Escherichia coli.

Another object of the invention is the use of a polynucleotide or of a recombinant vector of the invention as defined above, for preparing a modified host cell as defined above.

The introduction of the polynucleotide or of the recombinant vector according to the invention into the host cell to be modified can be carried out by any known method, such as, for example, transfection, infection, fusion, electroporation, microinjection or else biolistics.

Another object of the invention is the use of a cyclodipeptide synthetase of the invention as defined above, for producing cyclodipeptides and derivatives thereof.

According to an advantageous embodiment of said use of a cyclodipeptide synthetase of the invention as defined above, said CDS is used for producing the specific cyclodipeptides cyclo(Tyr-Tyr), cyclo(Tyr-Phe), cyclo(Tyr-Trp) and cyclo(Tyr-Ala).

In another aspect, the present invention relates to a method for the synthesis of cyclo(Tyr-Xaa) cyclodipeptides wherein Xaa is any amino acid, characterized in that it comprises:

(1) incubating two amino acids, Tyr and Xaa, which may be identical or different or their derivatives, under suitable conditions, with a cyclodipeptide synthetase of the invention as defined above, and

(2) recovering the cyclo(Tyr-Xaa) cyclodipeptides thus obtained.

The term “suitable conditions” is preferably intended to mean the appropriate conditions (concentrations, pH, buffer, temperature, time of reaction, etc. . . . ) under which the amino acids and the cyclodipeptide synthetase of the invention are incubated to allow the synthesis of said cyclodipeptides.

An example of an appropriate concentration of amino acids and cyclodipeptide synthetase is the following: amino acids (Tyr and Xaa) are at a concentration of 0.1 mM to 100 mM, preferably of 1 mM to 10 mM; the cyclodipeptide synthetases of the invention are at a concentration of 0.1 nM to 100 μM, preferably of 1 μM to 100 μM.

An example of an appropriate buffer is 100 mM Tris-HCl containing 150 mM NaCl, 10 mM ATP, 20 mM MgCl₂ supplemented with a soluble prokaryote cell extract.

Appropriate pH is ranging between 6 and 8, appropriate temperature is ranging between 20 and 40° C., and appropriate time is ranging between 12 and 24 hours.

Therefore, according to a preferred embodiment of carrying out said method, step (1) is performed in presence of appropriate aminoacids at a concentration between 0.1 mM to 100 mM, preferably of 1 mM to 10 mM, a cyclodipeptide synthetase according to the invention at a concentration between 0.1 nM and 100 μM, preferably of 1 μM to 100 μM, in a buffer at a pH between 6 and 8, and containing a soluble extract of prokaryote cells such as E. coli or Streptomyces cells which do not produce cyclodipeptide synthetase.

α,β-dehydrogenated cyclodipeptide derivatives may be obtained from the here above described cyclodipeptides, according to the method described in Gondry et al. (Eur. J. Biochem., 2001 precited) or in the International PCT Application WO 2004/000879.

For example, an amount of 5 10⁻³ units of CDO is added to the buffer used according to the method described above. One unit of CDO was defined as the amount catalyzing the formation of 1 μmol of cyclo(ΔPhe-His) per min under standard assay conditions (Gondry et al., Eur. J. Biochem., 2001, 268, 4918-4927).

Therefore according to a preferred embodiment of said method it comprises:

(1′) incubating two amino acids Tyr and Xaa, which may be identical or different under the suitable conditions as defined hereabove with a cyclodipeptide synthetase according to the invention and a purified CDO, and

(2′) recovering the α,β-dehydrogenated cyclodipeptides.

According to another preferred embodiment of the method of the synthesis of said cyclodipeptides or α,β-dehydrogenated derivatives thereby, a preliminary step (P) consisting in the use of a polynucleotide of the invention for synthesizing cyclodipeptide synthetases, is performed before step (1) or step (1′).

The methods of synthesis of cyclodipeptides and α,β-dehydrogenated derivatives thereof may be carried out in any suitable biological system, notably in a host such as, for example, a microorganism, for instance a bacterium such as Escherichia coli or Streptomyces lividans, or any known heterologous expression system using prokaryotes or eukaryotes as hosts, or even in acellular systems. According to a preferred embodiment, methods for the synthesis of cyclodipeptides and α,β-dehydrogenated derivatives thereof are carried out in a culture of the modified host cells of the invention expressing the cyclodipeptide synthetase of the invention.

Another object of the invention is the use of the modified host cell of the invention as defined above, for producing cyclo(Tyr-Xaa) cyclodipeptides, in particular cyclo(Tyr-Tyr), cyclo(Tyr-Phe), cyclo(Tyr-Trp) and cyclo(Tyr-Ala), and α,β-dehydrogenated derivatives thereof.

In another aspect, the present invention relates to a method for the synthesis of cyclo(Tyr-Xaa) cyclodipeptides, wherein Xaa is any amino acid, comprising the following steps:

(1′) culturing a host cell as defined here above in appropriate culture conditions for said host, and

(2′) recovering the cyclodipeptides from the culture medium.

α,β-dehydrogenated cyclodipeptides derivatives may be obtained from the here above described cyclodipeptides, according to the method described in Gondry et al. (Eur. J. Biochem., 2001, precited) or in the International PCT Application WO 2004/000879, in the following conditions:

(1′) culturing a host cell as defined here above in appropriate culture conditions for said host, and

(1″) incubating the culture medium containing the cyclodipeptides obtained from step (1′) with purified CDO, and

(2″) recovering the α,β-dehydrogenated cyclodipeptides derivatives obtained from step (1″) from the culture medium.

The conditions for using CDO are the same than those described above.

The recovering of the cyclodipeptides or of the α,β-dehydrogenated cyclodipeptides derivatives can be carried out directly from synthesis by means of liquid-phase extraction techniques or by means of precipitation, or thin-layer or liquid-phase chromatography techniques, in particular reverse-phase HPLC, or any method suitable for purifying peptides, one known to those skilled in the art.

Another object of the invention is the α,β-dehydrogenated cyclo(Tyr-Xaa) derivatives: cyclo(ΔTyr-Xaa), cyclo(Tyr-ΔXaa) and cyclo(ΔTyr-ΔXaa), wherein Xaa is any aminoacid.

According to a preferred embodiment of said derivatives they are selected in the group consisting of: cyclo(ΔTyr-Tyr), cyclo(ΔTyr-Phe), cyclo(ΔTyr-Trp), cyclo(ΔTyr-Ala), cyclo(Tyr-ΔPhe), cyclo(Tyr-ΔTrp), cyclo(Tyr-ΔAla), cyclo(ΔTyr-ΔTyr), cyclo(ΔTyr-ΔPhe), cyclo(ΔTyr-ΔTrp), cyclo(ΔTyr-ΔAla).

Besides the above provisions, the invention also comprises other provisions which would emerge from the following description, which refers to examples of implementation of the invention and also to the attached drawings, in which:

FIG. 1. (a) Structure of piperazine-2,5-dione cycle. The cis-amide bond is in bold. (b) Structure of cyclo(Tyr-Tyr). (c) Structure of cyclo(Tyr-Phe). (d) Structure of cyclo(Tyr-Trp). (e) Structure of cyclo(Tyr-Ala).

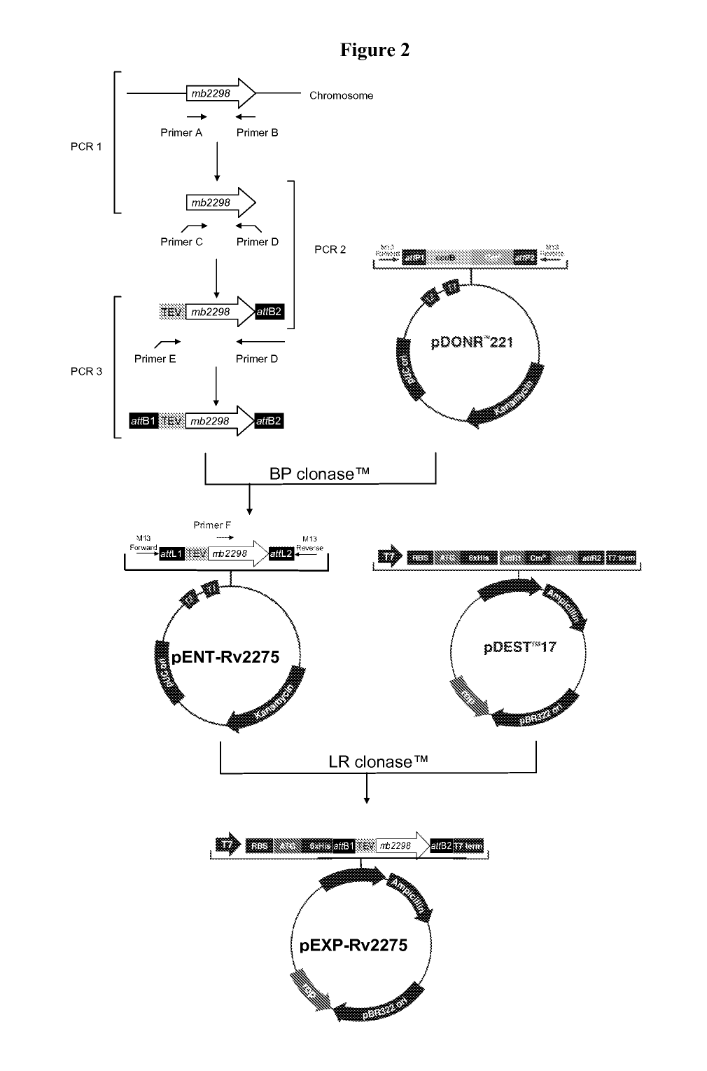

FIG. 2. The cloning strategy for the construction of the expression vector pEXP-Rv2275.

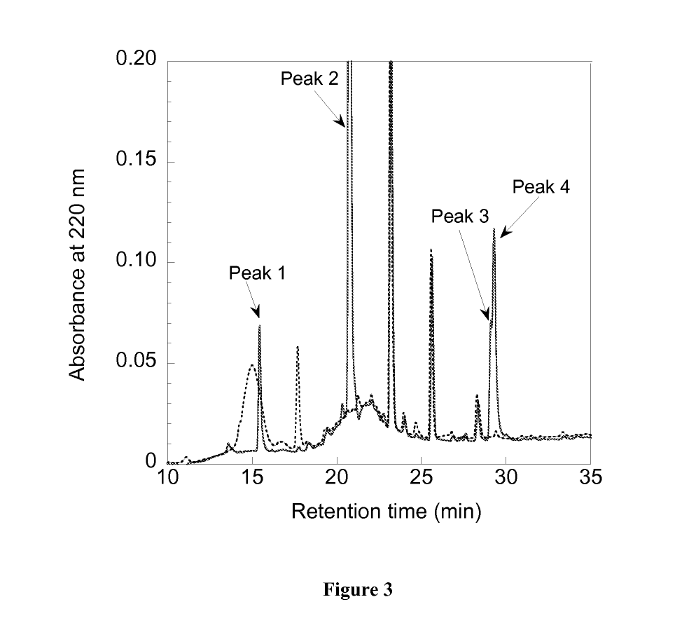

FIG. 3. HPLC analysis of the culture medium of E. coli BL21AI cells expressing the complete Rv2275 protein. Culture media of cells transformed with pEXP-Rv2275 (continuous line) and empty pQE60 (dotted line) were analyzed by RP-HPLC. Chromatograms were recorded at 220 nm.

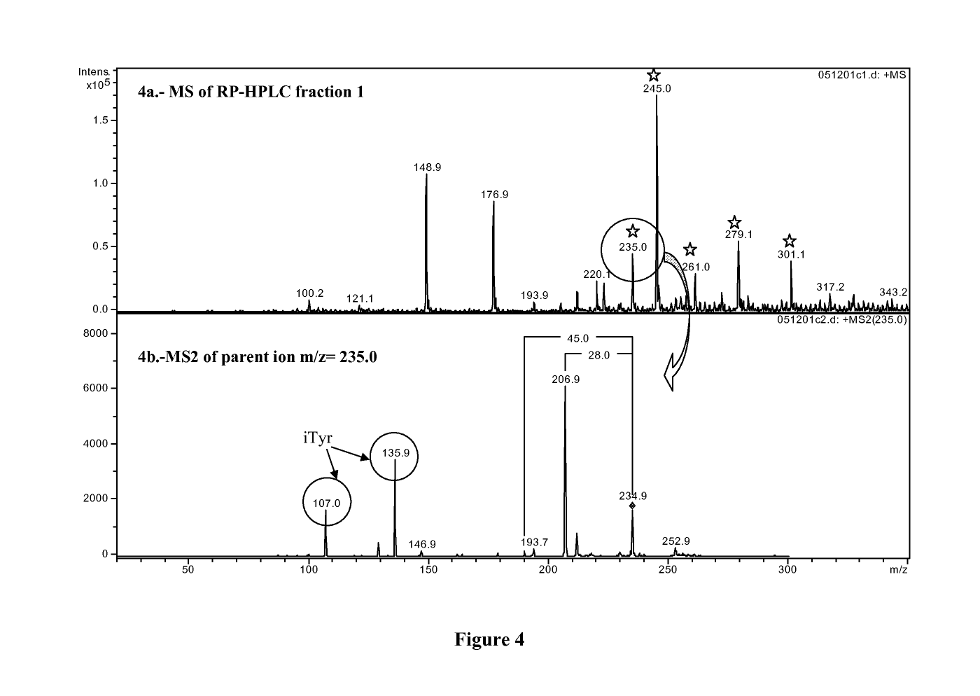

FIG. 4. MS (4a) and MSMS (4b) spectra of fraction 1 corresponding to the elution of peak 1 from the N-terminal tagged Rv2275 analysis (see FIG. 3). Collected fraction 1 was directly infused into the mass spectrometer and full scan MS acquired on line (FIG. 4a). m/z peaks quoted by star match to m/z of natural cyclodipeptides (Table 2) and then were selected for MSMS characterization under conditions described in Example 2. Only m/z peak at 235.0±10.1 displays a sequence of fragmentation typical to cyclodipeptides: neutral losses of 28 and 45 and production of the so-called immonium and related ion of tyrosine (hereinafter referred to as “iTyr”) as shown in the daughter ion spectrum in FIG. 4b.

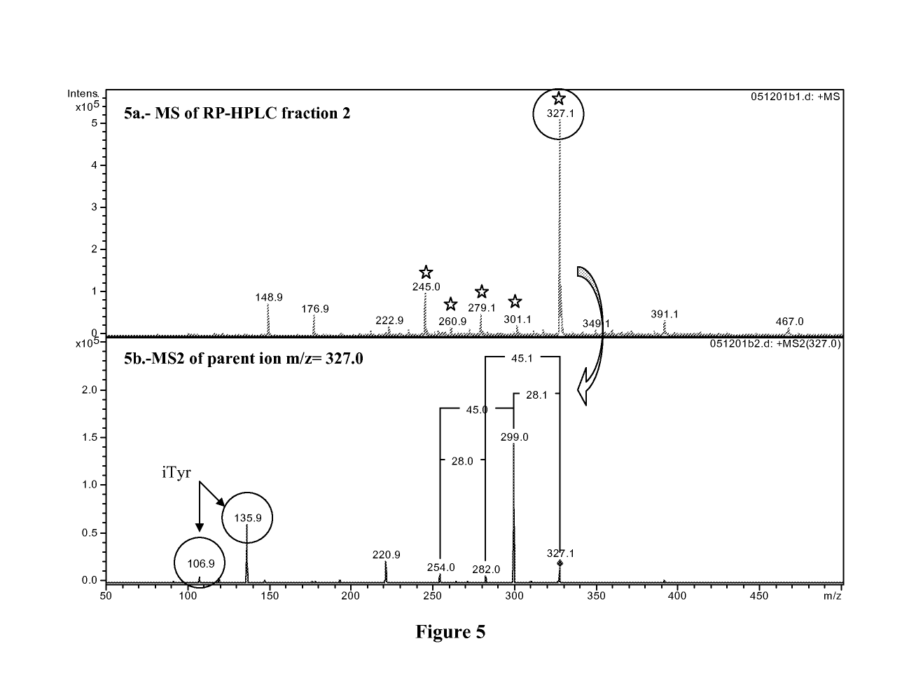

FIG. 5. MS (5a) and MSMS (5b) spectra of fraction 2 corresponding to the elution of peak 2 from the N-terminal tagged Rv2275 analysis (see FIG. 3). Collected fraction 2 was directly infused into the mass spectrometer and full scan MS acquired on line (FIG. 5a). Main peak with m/z at 327.0±0.1 was selected for structural characterization by MSMS fragmentation under conditions described in Example 2. Daughter ion spectrum displays a sequence of neutral losses of 28 and 45 and m/z peak at 136.0±0.1, which matches to immonium ion of tyrosine (hereinafter referred to as “iTyr”) (FIG. 5b).

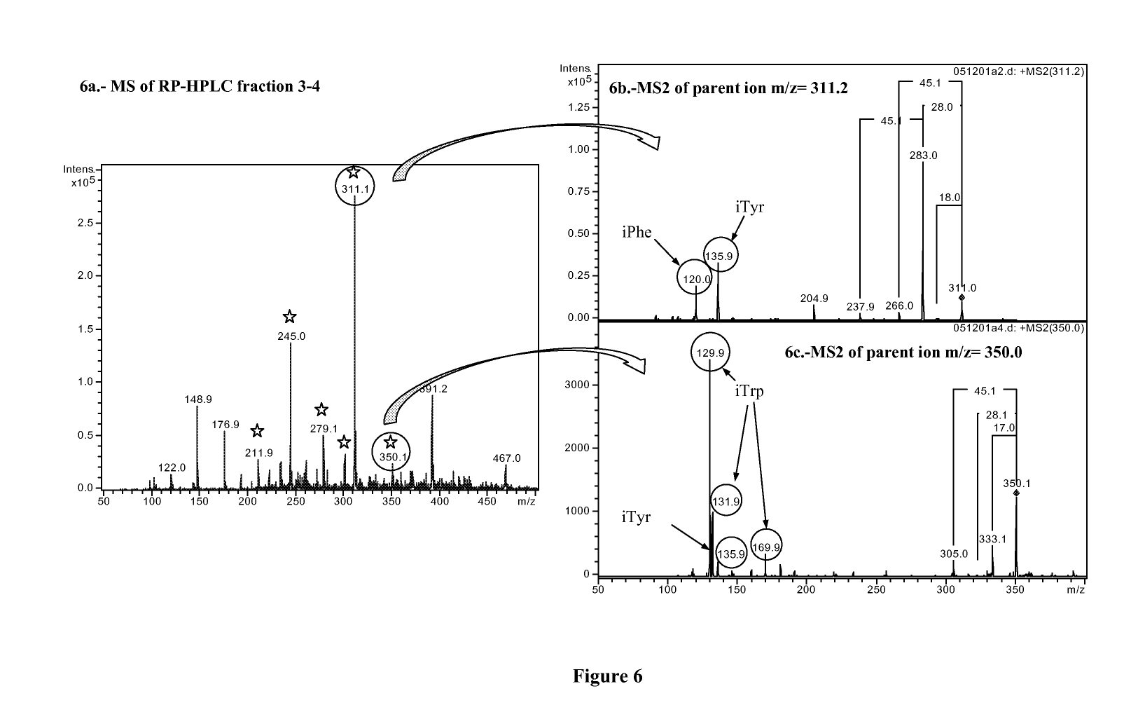

FIG. 6. MS (6a) and MSMS (6b and 6c) spectra of fractions 3-4 corresponding to the coelution of peaks 3 and 4 from the N-terminal tagged Rv2275 analysis (see FIG. 3). Collected fractions 3-4 were directly infused into the mass spectrometer and full scan MS acquired on line (FIG. 6a). Daughter ions spectra of the designated cyclodipeptides matched m/z peak (encircled peak in the MS spectra) were obtained under conditions described in Example 2. Immonium ion and related ion of tyrosine, phenylalanine and tryptophane are respectively referred to as “iTyr”, “iPhe” and “iTrp”.

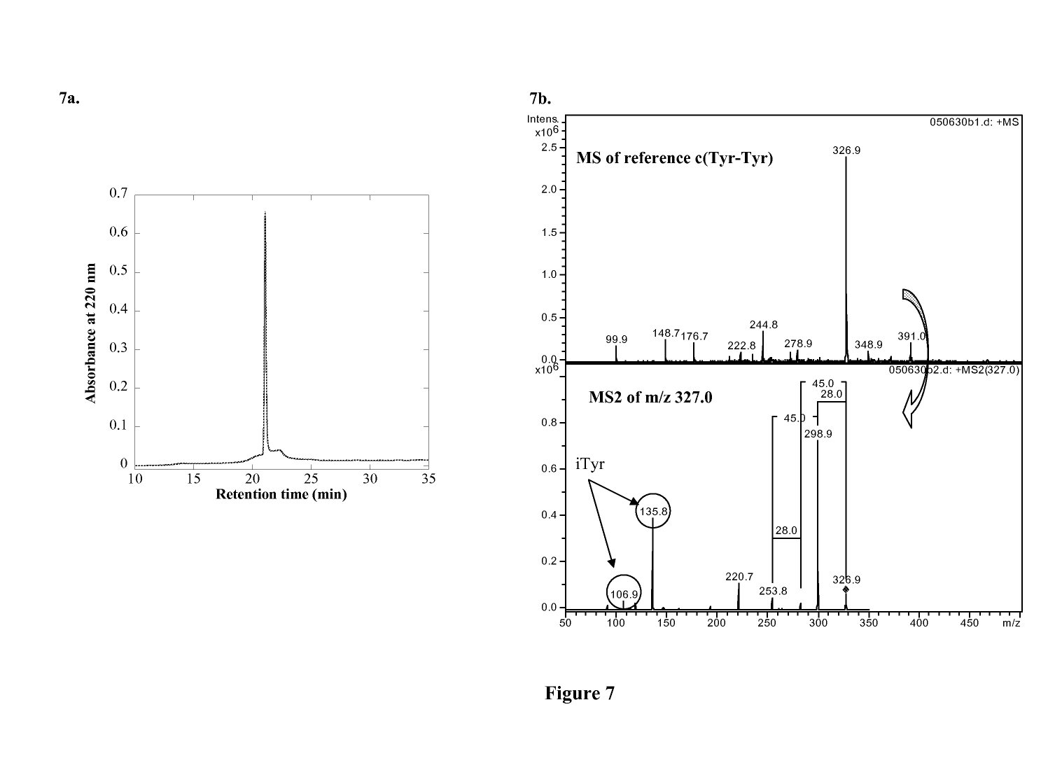

FIG. 7. HPLC analysis (7a), MS and MSMS spectra (7b) of commercial cyclo(Tyr-Tyr). The chromatogram was recorded at 220 nm. MS and daughter ions spectra of cyclo(Tyr-Tyr) at m/z 327.0±0.1 were obtained under conditions described in Experimental Methods. Immonium and related ions of tyrosine are hereinafter referred to as “iTyr”.

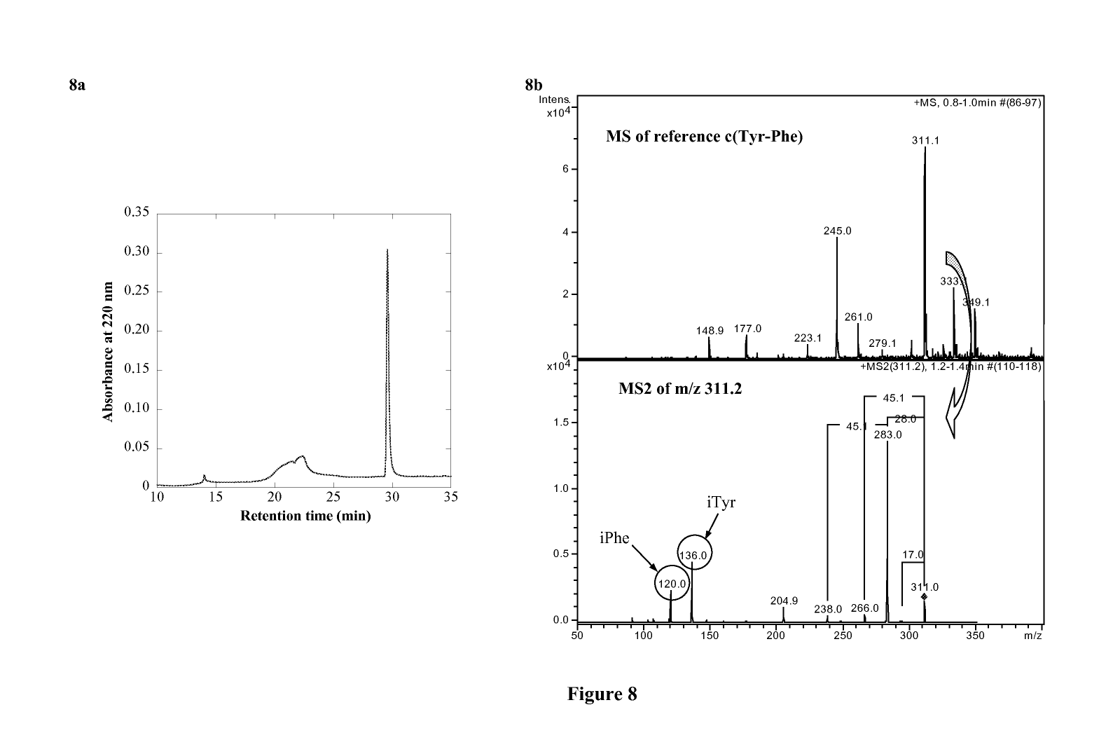

FIG. 8. HPLC analysis (8a), MS and MSMS spectra (8b) of synthesized cyclo(Tyr-Phe). The chromatogram was recorded at 220 nm. MS and daughter ions spectra of cyclo(Tyr-Phe) at m/z 311.2±0.1 were obtained under conditions described in Experimental Methods. Immonium ions of tyrosine and phenylalanine are respectively referred to as “iTyr” and “iPhe”.

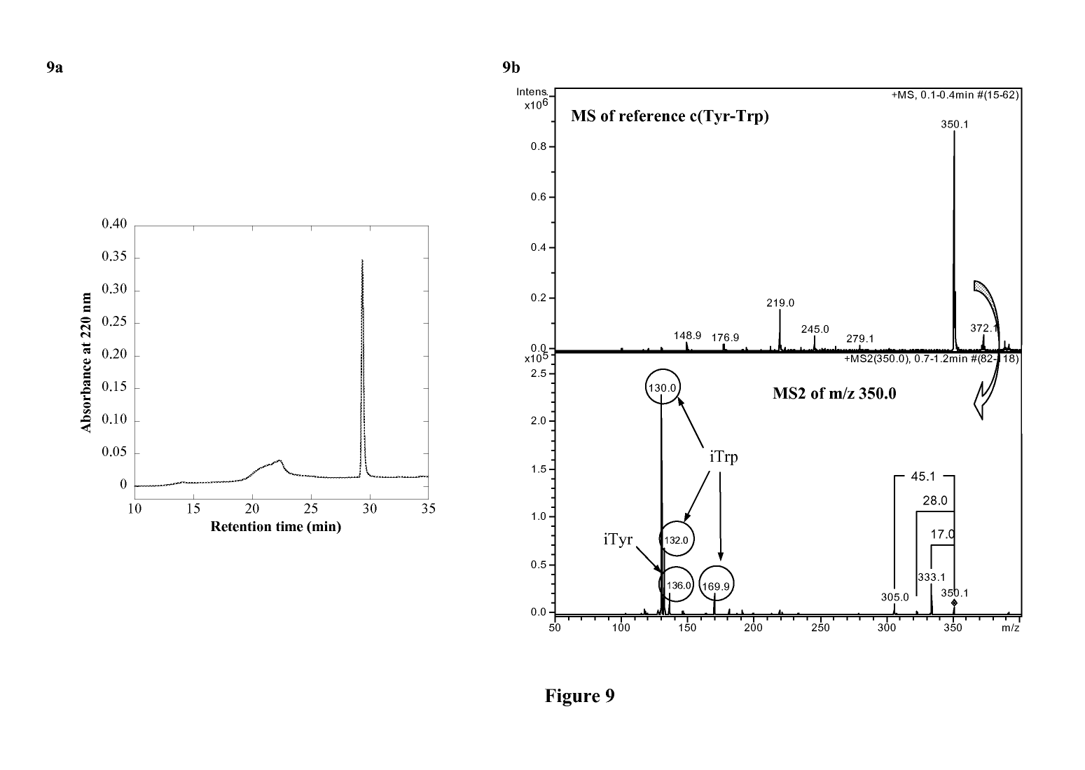

FIG. 9. HPLC analysis (9a), MS and MSMS spectra (9b) of synthesized cyclo(Tyr-Trp). The chromatogram was recorded at 220 nm. MS and daughter ions spectra of cyclo(Tyr-Trp) at m/z 350.0±0.1 were obtained under conditions described in Experimental Methods. Immonium and related ions of tyrosine and tryptophan are respectively referred to as “iTyr” and “iTrp”.

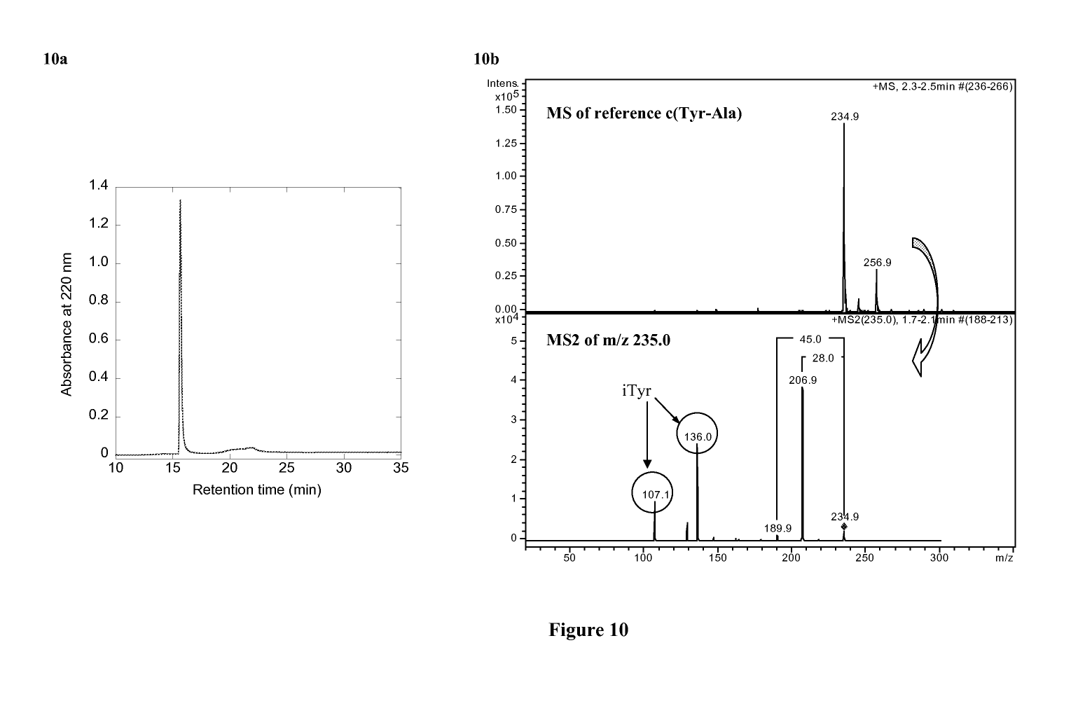

FIG. 10. HPLC analysis (10a), MS and MSMS spectra (10b) of synthesized cyclo(Tyr-Ala). The chromatogram was recorded at 220 nm. MS and daughter ions spectra of cyclo(Tyr-Ala) at m/z 235.0±0.1 were obtained under conditions described in Experimental Methods. Immonium ion of tyrosine is hereinafter referred to as “iTyr”. Immonium ion of Ala is not detected.

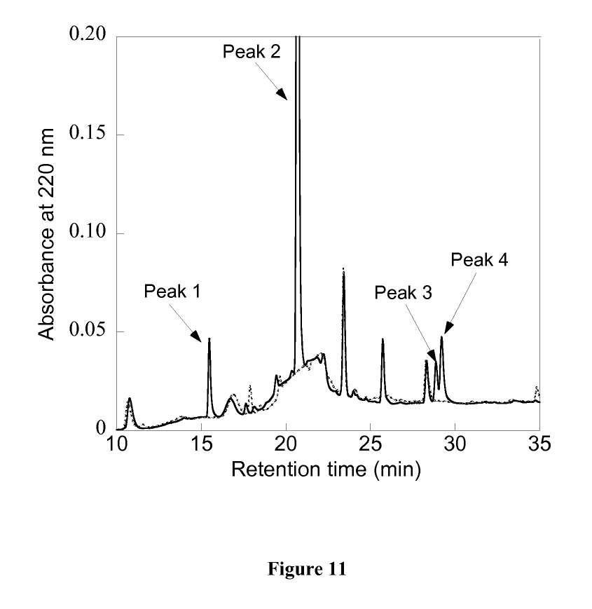

FIG. 11. HPLC analysis of the culture medium of E. coli M15[pREP4] cells expressing the truncated Rv2275 protein. Culture media of cells transformed with pQE60-Rv2275C (continuous line) and pQE60 (dotted line) were analyzed by RP-HPLC. Chromatograms were recorded at 220 nm.

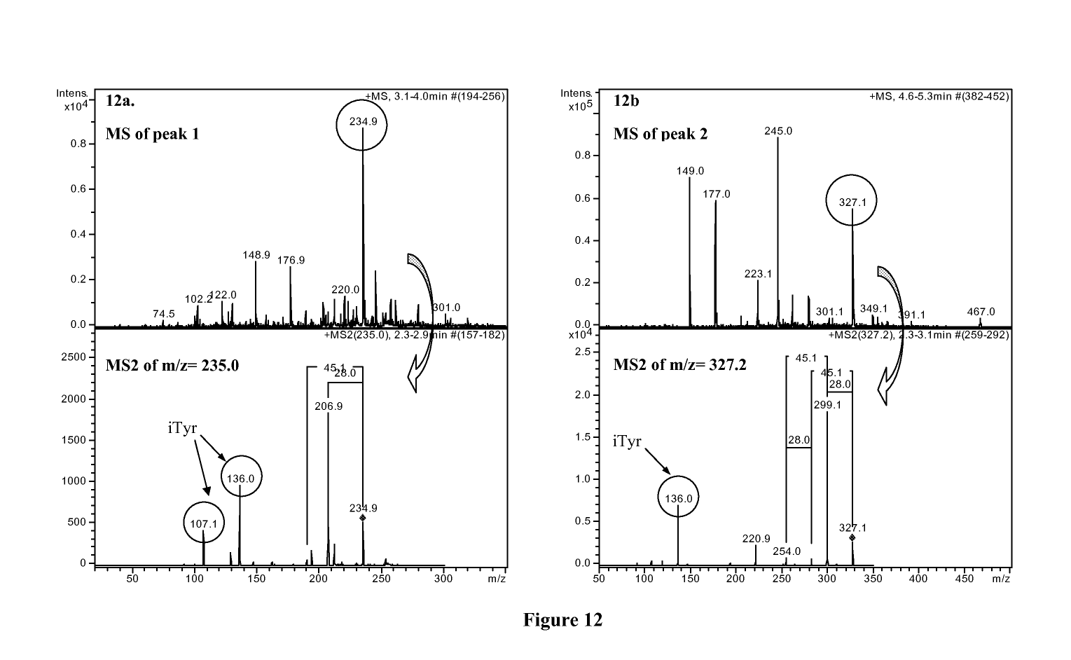

FIG. 12. MS and MSMS spectra of peak 1 (FIG. 12a) and of peak 2 (FIG. 12b) obtained from the truncated Rv2275 analysis (see FIG. 11). Collected fractions were directly infused into the mass spectrometer and full scan MS acquired on line (top spectra). Daughter ions spectra (bottom spectra) of the designated cyclodipeptides matched m/z peak (encircled peak in the MS spectra) were obtained under conditions described in Experimental Methods. Immonium and related ions of tyrosine are hereinafter referred to as “iTyr”.

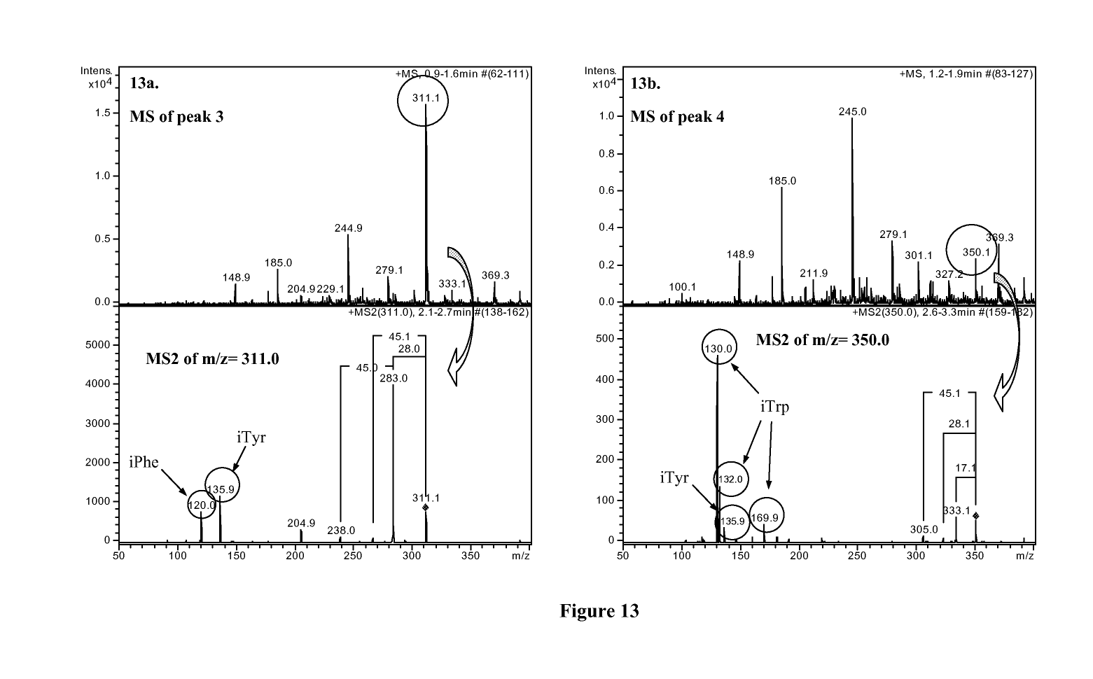

FIG. 13. MS and MSMS spectra of peak 3 (FIG. 13a) and of peak 4 (FIG. 13b) obtained from the truncated Rv2275 analysis (see FIG. 11). Collected fractions were directly infused into the mass spectrometer and full scan MS acquired on line (top spectra). Daughter ions spectra (bottom spectra) of the designated cyclodipeptides matched m/z peak (encircled peak in the MS spectra) were obtained under conditions described in Experimental Methods. Immonium and related ions of tyrosine, phenylalanine and tryptophan are respectively referred to as “iTyr” and “iPhe” and “iTrp”.

The following examples illustrate the invention but in no way limit it.

### EXAMPLE 1

### Construction of an Escherichia coli Expression Vector Encoding the Rv2275 Protein as a N-Terminal His₆-Tagged Fusion

The expression vector encoding Rv2275 was constructed using the Gateway™ cloning technology (Invitrogen). It was designed to express Rv2275 as a cytoplasmic fusion protein carrying at its N-terminus end a (His)₆ tag, the translated sequence of the attB recombination site (necessary for cloning) and the TEV protease cleavage site, resulting in a N-terminal extension of 29 residues, i.e. MSYYHHHHHHLESTSLYKKAGFENLYFQG (SEQ ID NO: 18). As the gene encoding Rv2275 is conserved in several strains of the Mycobacterium tuberculosis complex (M. tuberculosis, M. bovis), we used for the construction of this expression vector the chromosomal DNA from Mycobacterium bovis BCG Pasteur that carries the mb2298 gene (gi:31793454) 100% identical to the Rv2275 gene of Mycobacterium tuberculosis H37Rv (gi:15609412).

The whole cloning strategy is shown in FIG. 2. First, the attB-flanked DNA suitable for recombinational cloning and encoding the Rv2275 protein was obtained after three successive PCR. The mb2298 gene was amplified in the first PCR (PCR 1 in FIG. 2) using Mycobacterium bovis BCG Pasteur genomic DNA as a template and primers A and B (Table I). The PCR conditions were one initial denaturation step at 97° C. for 4 minutes followed by 25 cycles at 94° C. for one minute, 54° C. for one minute, 72° C. for 2 minutes, and one final extension step at 72° C. for 10 minutes. The reaction mixture (50 μl) comprised 1 μl of chromosomal DNA (25 ng/μl), 0.3 μl of each primer solution at 100 μM, 5 μl of 10×Pfu DNA polymerase buffer with MgSO₄ (provided by the Pfu DNA polymerase supplier), 0.1 μl of a mix of dNTPs 10 mM each and 1 μl of Pfu DNA polymerase (2.5 U/μl; Fermentas). The PCR product (herein after referred to as “PCR product 1”) was then purified using the “GFX PCR DNA and Gel Band Purification” kit (Amersham Biosciences) after electrophoresis in 1% agarose gel (Sambrook et al., Molecular Cloning: A Laboratory manual, 2001, New York). The second PCR (PCR 2 in FIG. 2) enabled the addition of the sequence encoding the TEV protease cleavage site to the PCR product 1 5′-terminus and that of the attB2 encoding sequence to the 3′-terminus. PCR conditions were one initial denaturation step at 95° C. for 5 minutes followed by 30 cycles at 95° C. for 45 seconds, 50° C. for 45 seconds, 72° C. for 1.5 minutes, and one final extension step at 72° C. for 10 minutes. The reaction mixture (50 μl) comprised 5 ng PCR product 1, 0.4 μM primers C and D (see Table I), 2.5 units Expand High Fidelity Enzyme mix (Roche), 1× Expand High Fidelity buffer with 1.5 mM MgCl₂ (Roche) and 200 μM each dNTP. After electrophoresis in 1% agarose gel and purification with the QIAquick Gel Extraction kit (Qiagen), the PCR product (hereinafter referred to as “PCR product 2”) was used for the third PCR (PCR 3 in FIG. 2) that enabled the addition to the PCR product 2 5′-terminus of the attB1 encoding sequence. PCR was carried out as described above using 5 ng PCR product 2 as a template and 0.4 μM primers E and D (see Table I). The resulting PCR product (hereinafter referred to as “PCR product 3”) was purified as previously described.

Second, the attB-flanked PCR product 3 was recombined with the pDONR™221 donor vector (Invitrogen) in BP Clonase™ reaction to generate the entry vector pENT-Rv2275. pENT-Rv2275 was sequenced between the two-recombination sites using ABI PRISM 310 Genetic Analyzer (Applied Biosystem) and primers M13 forward, M13 reverse and F (see Table I). pENT-Rv2275 and the commercial destination vector pDEST-17 (Invitrogen) were used in LR Clonase™ subcloning reaction to generate the expression vector pEXP-Rv2275 (SEQ ID NO: 19) following the supplier recommendations.

TABLE I
Primers used to construct the expression vector pEXP-Rv2275.

| Name | Corresponding sequence (5′ to 3′) |
|---|---|
| A | CCGTCCCTATGGTCCAAGGAAAACAATGTCATACG (SEQ ID NO: 7) |
| B | GCAAGCAATAACGGCGGGGCTCCCATCAGGGGTA (SEQ ID NO: 8) |
| C | GGCTTCGAGAATCTTTATTTTCAGGGCTCATACGTGGCTGCC (SEQ ID NO: 9) |
| D | GGGGACCACTTTGTACAAGAAAGCTGGGTCCTTATTCGGCGGGGCTC (SEQ ID NO: 10) |
| E | GGGGACAAGTTTGTACAAAAAAGCAGGCTTCGAGAATCTTTATTTTC (SEQ ID NO: 11) |
| F | TCGGCCATTCACCCAACAAT (SEQ ID NO: 12) |
| M13 Forward | GTAAACGACGGCCAG (SEQ ID NO: 13) |
| M13 Reverse | CAGGAAACAGCTATGAC (SEQ ID NO: 14) |

The recombination mixture was used for transformation of E. coli DH5α chemically competent cells and a positive clone was selected after analysis by colony PCR. The 50 μl reaction mix comprised a small amount of colony as a template, 200 μM each dNTP, 0.2 μM primer M13 forward and M13 reverse, 1× ThermoPol Reaction Buffer (New England Biolabs) and 1.25 unit Taq DNA Polymerase (New England Biolabs). The PCR conditions used were the following: one initial denaturation step at 95° C. for 5 minutes followed by 30 cycles at 92° C. for 30 seconds, 50° C. for 30 seconds, 72° C. for 2 minutes. Plasmid DNA was isolated from positive clones using the Wizard DNA Purification System (Promega) and conserved at −20° C.

### EXAMPLE 2

### Expression of Rv2275 in Escherichia coli Cytoplasm Leads to the Synthesis of Cyclodipeptides

The expression of Rv2275 was performed by cultivating E. coli cells harboring the plasmid pEXP-Rv2275 in minimal medium. This medium contains all the elements of the M9 minimal medium (6 g/l Na₂HPO₄, 3 g/l KH₂PO₄, 0.5 g/l NaCl, 1 g/l NH₄Cl, 1 mM MgSO₄, 0.1 mM CaCl₂, 1 μg/ml thiamine and 0.5% glucose or glycerol) (Sambrook et al., aforementioned) plus 1 ml of a vitamins solution and 2 ml of an oligoelements solution per liter of minimal medium. Vitamins solution contains 1.1 mg/l biotin, 1.1 mg/l folic acid, 110 mg/l para-aminobenzoic acid, 110 mg/l riboflavin, 220 mg/l pantothenic acid, 220 mg/l pyridoxine-HCl, 220 mg/l thiamine and 220 mg/l niacinamide in 50% ethanol. Oligoelements solution was made by diluting a FeCl₂-containing solution 50 fold in H₂O. The FeCl₂-containing solution contains for 100 ml: 8 ml concentrated HCl, 5 g FeCl₂.4H₂O, 184 mg CaCl₂.2H₂O, 64 mg H₃BO₃, 40 mg MnCl₂.4H₂O, 18 mg CoCl₂.6H₂O, 4 mg CuCl₂.2H₂O, 340 mg ZnCl₂, 605 mg Na₂MoO₄.2H₂O.

Recombinant expression of Rv2275 from the plasmid pEXP-Rv2275 was made using E. coli BL21AI cells (Invitrogen). 50 μl chemically competent cells were transformed with 20 ng plasmid using standard heat-shock procedure (Sambrook et al., aforementioned). BL21AI cells were also transformed by pQE60 in order to make the CDS-non producing control culture. After 1 h outgrowth in SOC medium (Sambrook et al., aforementioned) at 37° C., bacteria of the two transformation mix were spread on LB plates containing 200 μg/ml ampicillin and incubated overnight at 37° C. A few colonies were picked up to inoculate M9 liquid medium supplemented with vitamins and oligoelements solutions containing 0.5% glucose and 200 μg/ml ampicillin. After overnight incubation at 37° C. with shaking, 500 μl of each starter culture were used to inoculate 25 ml M9 minimal medium supplemented with vitamins and oligoelements solutions containing 0.5% glycerol and 200 μg/ml ampicillin. Bacteria were grown at 37° C. until OD₆₀₀˜0.5 and 0.02% L-arabinose was added. Cultures were continued at 20° C. for 24 h. Cultures supernatants were collected after centrifugation at 3,000 g for 20 minutes and kept at −20° C.

### Detection of Cyclodipeptide Derivatives by HPLC Analysis.

The formation of cyclodipeptides has been investigated by analyzing the culture supernatant of E. coli cells expressing Rv2275, as previously reported for the culture supernatant of E. coli cells expressing AlbC (Lautru et al., Chem. Biol., 2002, 9, 1355-1364).

Culture supernatants (200 μl) were acidified down to pH=3 with concentrated trifluoroacetic acid and then analyzed by HPLC. Samples were loaded onto a C18 column (4.6×250 mm, 5 μm, 300 Å, Vydac) and eluted with a linear gradient from 0% to 55% acetonitrile/deionized water with 0.1% trifluoroacetic acid for 60 min (flow-rate, 1 ml/min). The elution was monitored between 220 and 500 nm using a diode array detector.

HPLC analysis at 220 nm of the culture supernatant of E. coli cells expressing N-terminal tagged Rv2275 showed two resolved peaks, namely peak 1 and peak 2, and two poorly-resolved peaks, namely peak 3 and peak 4, that were not found in the supernatant of a culture of cells which did not express Rv2275 (empty pQ60 used) (FIG. 3). These four peaks hence corresponded to products whose synthesis was directly linked to the expression of Rv2275 in E. coli. Peak 2 was the major peak and it was characterized by a retention time of 21.3 min and an absorption band centered at around 275 nm. Peak 1 was the minor peak and it was characterized by a retention time of 15.9 min and an absorption band centered at around 275 nm. Peaks 3 and 4 are not completely resolved as they displayed very close retention times. Peak 3 was characterized by a retention time of 29.6 min and an absorption band centered at around 277 nm with a shoulder at 288 nm. Peak 4 was characterized by a retention time of 29.8 min and an absorption band centered at around 275 nm. The four peaks were collected for further analysis by mass spectrometry.

### Identification of Cyclodipeptide Derivatives by MS and MSMS Analysis.

HPLC-eluted fractions from culture supernatants (see above) were collected and analyzed by mass spectrometry using an ion trap mass analyzer Esquire HCT equipped with an orthogonal Atmospheric Pressure Interface-ElectroSpray Ionization (AP-ESI) source (Bruker Daltonik GmbH, Germany). The samples were directly infused into the mass spectrometer at a flow rate of 3 μl/min by means of a syringe pump. Nitrogen served as the drying and nebulizing gas while helium gas was introduced into the ion trap for efficient trapping and cooling of the ions generated by the ESI as well as for fragmentation processes. Ionization was carried out in positive mode with a nebulizing gas set at 9 psi, a drying gas set at 5 μl/min and a drying temperature set at 300° C. Ionization and mass analyses conditions (capillary high voltage, skimmer and capillary exit voltages and ions transfer parameters) were set for an optimal detection of compounds in the range of cyclodipeptides masses between 100 and 400 m/z. For structural characterization by mass fragmentations, an isolation width of 1 mass unit was used for isolating the parent ion. Fragmentation amplitude was tuned until at least 90% of the isolated precursor ion was fragmented. Full scan MS and MSMS spectra were acquired using EsquireControl software and all data were processed using DataAnalysis software.

Commercial cyclodipeptides (Sigma and Bachem) or chemically-synthesized cyclodipeptides (as described in Jeedigunta et al., Tetrahedron, 2000, 56, 3303-3307) were used as standard for mass and HPLC analyses.

MS spectra of the eluted fractions corresponding to the N-terminal tagged Rv2275, hereinafter referred to as “fraction 1” for the elution of peak 1, “fraction 2” for the elution of peak 2 and “fraction 3-4” for the elution of both peaks 3 and 4 were respectively shown in FIGS. 4a, 5a & 6a. These spectra showed heterogeneous masses and rather low intensity m/z peaks. This might be due to the difficulty of the cyclodipeptides potentially produced by Rv2275 to be ionized by electrospray ionisation. Starting from MS spectra, we compared all significant m/z values (Signal/Noise ratio>5) to expected mass values of natural cyclodipeptides (quoted in Table II).

TABLE II
Calculated monoisotopic mass (m/z) values of natural cyclodipeptides under positive mode of ESI-MS.

| m/z | | | | | | | | | | | | | | | | | | | | |
|---|---|---|---|---|---|---|---|---|---|---|---|---|---|---|---|---|---|---|---|---|
| of | | | | | | | | | | | | | | | | | | | | |
| AA | | | | | | | | | | | | | | | | | | | | |
| resi- | | | | | | | | | | | | | | | | | | | | |
| due | Gly | Ala | Ser | Pro | Val | Thr | Cys | Ile | Leu | Asn | Asp | Gln | Lys | Glu | Met | His | Phe | Arg | Tyr | Trp |
| Gly | 115.1 | 129.1 | 145.1 | 155.2 | 157.2 | 159.2 | 161.2 | 171.2 | 171.2 | 172.2 | 173.1 | 186.2 | 186.2 | 187.2 | 189.3 | 195.2 | 205.2 | 214.2 | 221.2 | 244.3 |
| Ala | | 143.2 | 159.2 | 169.2 | 171.2 | 173.2 | 175.2 | 185.2 | 185.2 | 186.2 | 187.2 | 200.2 | 200.3 | 201.2 | 203.3 | 209.2 | 219.3 | 228.3 | 235.3 | 258.3 |
| Ser | | | 175.2 | 185.2 | 187.2 | 189.2 | 191.2 | 201.2 | 201.2 | 202.2 | 203.2 | 216.2 | 216.3 | 217.2 | 219.3 | 225.2 | 235.3 | 244.3 | 251.3 | 274.3 |
| Pro | | | | 195.2 | 197.3 | 199.2 | 201.3 | 211.3 | 211.3 | 212.2 | 213.2 | 226.3 | 226.3 | 227.2 | 229.3 | 235.3 | 245.3 | 254.3 | 261.3 | 284.3 |
| Val | | | | | 199.3 | 201.2 | 203.3 | 213.3 | 213.3 | 214.2 | 215.2 | 228.3 | 228.3 | 229.2 | 231.3 | 237.3 | 247.3 | 256.3 | 263.3 | 286.3 |
| Thr | | | | | | 203.2 | 205.2 | 215.3 | 215.3 | 216.2 | 217.2 | 230.2 | 230.3 | 231.2 | 233.3 | 239.2 | 249.3 | 258.3 | 265.3 | 288.3 |
| Cys | | | | | | | 207.3 | 217.3 | 217.3 | 218.2 | 219.2 | 232.3 | 232.3 | 233.3 | 235.3 | 241.3 | 251.3 | 260.3 | 267.3 | 290.4 |
| Ile | | | | | | | | 227.3 | 227.3 | 228.3 | 229.3 | 242.3 | 242.3 | 243.3 | 245.4 | 251.3 | 261.3 | 270.4 | 277.3 | 300.4 |
| Leu | | | | | | | | | 227.3 | 228.3 | 229.3 | 242.3 | 242.3 | 243.3 | 245.4 | 251.3 | 261.3 | 270.4 | 277.3 | 300.4 |
| Asn | | | | | | | | | | 229.2 | 230.2 | 243.2 | 243.3 | 244.2 | 246.3 | 252.2 | 262.3 | 271.3 | 278.3 | 301.3 |
| Asp | | | | | | | | | | | 231.2 | 244.2 | 244.3 | 245.2 | 247.3 | 253.2 | 263.3 | 272.3 | 279.3 | 302.3 |
| Gln | | | | | | | | | | | | 257.3 | 257.3 | 258.2 | 260.3 | 266.3 | 276.3 | 285.3 | 292.3 | 315.3 |
| Lys | | | | | | | | | | | | | 257.3 | 258.3 | 260.4 | 266.3 | 276.4 | 285.4 | 292.4 | 315.4 |
| Glu | | | | | | | | | | | | | | 259.2 | 261.3 | 267.3 | 277.3 | 286.3 | 293.3 | 316.3 |
| Met | | | | | | | | | | | | | | | 263.4 | 269.3 | 279.4 | 288.4 | 295.4 | 318.4 |
| His | | | | | | | | | | | | | | | | 275.3 | 285.3 | 294.3 | 301.3 | 324.4 |
| Phe | | | | | | | | | | | | | | | | | 295.4 | 304.4 | 311.4 | 334.4 |
| Arg | | | | | | | | | | | | | | | | | | 313.4 | 320.4 | 343.4 |
| Tyr | | | | | | | | | | | | | | | | | | | 327.4 | 350.4 |
| Trp | | | | | | | | | | | | | | | | | | | | 373.4 |

In a second step, MSMS experiments were performed in order to elucidate the chemical structure of the compounds whose m/z values matched to that of cyclodipeptides.

As already experimented on different commercial or home-made synthetic cyclodipeptides and also observed on cyclodipeptides daughter ions spectra published elsewhere (Chen et al., Eur. Food Research technology, 2004, 218, 589-597; Stark et al., J. Agric. Food Chem., 2005, 53, 7222-7231), cyclodipeptide derivatives are fragmented following a characteristic pattern: (i) a sequence of neutral losses which results from cleavages of the diketopiperazine ring on either sides of the carbonyl group (loss of 28 uma corresponding to the departure of C═O group) or of the amido group (loss of 45 corresponding to CONH₃) and (ii) the presence of m/z peaks of the so-called immonium ions and of their related ions (Roepstorff et al., Biomed. Mass Spectrom., 1984, 11, 601; Johnson et al., Anal. Chem., 1987, 59, 2621-2625) which enable to identify aminoacyl residues (Table III).

TABLE III
Immonium and related ion masses m/z used for the
identification of the cyclodipeptides according
to Falick, A. M. et al., J. Am. Soc.
Mass Spectrom., 1993, 4, 882-893 and
Papayannopoulos, I. A., Mass Spectrom.
Rev., 1995, 14, 49-73.

| Residue | Immonium ion* | Related ions* |
|---|---|---|
| Gly | 30 | |
| Ala | 44 | |
| Ser | 60 | |
| Pro | 70 | |
| Val | 72 | 41, 55, 69 |
| Thr | 74 | |
| Cys | 76 | |
| Ile | 86 | 44, 72 |
| Leu | 86 | 44, 72 |
| Asn | 87 | 70 |
| Asp | 88 | 70 |
| Gln | 101 | 56, 84, 129 |
| Lys | 101 | 70, 84, 112, 129 |
| Glu | 102 | |
| Met | 104 | 61 |
| His | 110 | 82, 121, 123, 138, 166 |
| Phe | 120 | 91 |
| Arg | 129 | 59, 70, 73, 87, 100, 112 |
| Tyr | 136 | 91, 107 |
| Trp | 159 | 77, 117, 130, 132, 170, 171 |

*Bold face indicates strong signals, italic indicates weak.

The matched cyclodipeptide m/z peaks (marked with a star in FIGS. 4a, 5a & 6a) were then subjected individually to MSMS fragmentations and all the obtained daughter ions spectra were screened for cyclodipeptide fragmentation pattern.

MS analysis of fraction 1 showed five m/z peaks whose m/z value matches to a natural cyclodipeptide (FIG. 4a). However, only one (235.0±0.1) after MSMS fragmentation gave rise to a typical pattern of cyclodipeptide showing neutral losses of 28 and 45 and the appearance of m/z peaks corresponding to immonium (136±0.1) and related ion (107±0.1) of tyrosine (FIG. 4b). According to masses shown in Table II, the only cyclodipeptide containing a tyrosyl residue with a m/z of 235 is the cyclo(Tyr-Ala). Hence, the compound eluted at a retention time of 15.9 min was shown to be cyclo(Tyr-Ala).

MS analysis of fraction 2 showed five m/z peaks whose m/z value matches to a natural cyclodipeptide (FIG. 5a). However, only the major peak at 327.1±0.1 presented a fragmentation pattern typical of a cyclodipeptide (FIG. 5b). Compared to m/z values of natural cyclodipeptides of Table 2, this m/z matches to cyclo(Tyr-Tyr). In addition, daughter ions spectrum of this m/z 327.1±0.1 showed the presence of immonium ion of tyrosine (FIG. 5b). This result indicates that Rv2275 product eluted in the fraction 2 at 21.3 min is cyclo(Tyr-Tyr).

MS analysis of fraction 3-4 showed two peaks both matching expected m/z values for cyclodipeptides and presenting a fragmentation pattern typical of a cyclodipeptide (FIG. 6): a peak with a m/z=311.1±0.1 and a smaller one with a m/z=350.0±0.1 (FIG. 6a). Structural characterization by MSMS fragmentation identified the m/z peak at 311.1±0.1 as cyclo(Tyr-Phe) in reference to detected immonium ions of tyrosine (m/z=136.0±0.1) and of phenylalanine (m/z=120.0±0.1) (FIG. 6b). MSMS analysis of the tiny m/z peak at 350.0±0.1 produced immonium ion of tyrosine (m/z=136.0±0.1) and three of the immonium related ions of tryptophan (m/z=130.0±0.1, 132.0±0.1 and 170.0±0.1) (FIG. 6c). Peak with a m/z at 350.0±0.1 was then attributed to cyclo(Tyr-Trp).

### EXAMPLE 3

### Expression of Rv2275 in Escherichia coli Cytoplasm Leads to the Synthesis of Cyclo(Tyr-Xaa)

The previously presented MS analyses strongly suggest that the compounds identified in culture supernatants of E. coli cells producing Rv2275 were cyclo(Tyr-Ala), cyclo(Tyr-Tyr), cyclo(Tyr-Phe) and cyclo(Tyr-Trp). To confirm these identifications, the commercial cyclo(Tyr-Tyr) and cyclo(Tyr-Trp) and the chemically-synthesized cyclo(Tyr-Phe) and cyclo(Tyr-Ala) were used as references for both HPLC analyses and MSMS experiments. All of these standard cyclodipeptides display the same chromatographic and mass features detected above. Indeed, the retention time of the reference cyclo(Tyr-Tyr) (see FIG. 7a) is identical to that of the peak 2 of the culture supernatant (FIG. 3). Second, the reference cyclo(Tyr-Tyr) was submitted to MS and MSMS analysis and the resulting fragmentation pattern (FIG. 7b) was found similar to that obtained in FIG. 5b. Standard cyclo(Tyr-Phe) elutes at a retention time identical to that obtained for the poorly-resolved peak 4 (FIG. 8a compared to FIG. 3). The MS and MSMS features of this standard cyclodipeptide (FIG. 8b) are identical to that obtained for peak 4 (FIG. 6b). In the same way, RP-HPLC and MS analyses on standard cyclo(Tyr-Trp) (FIGS. 9a and 9b) and cyclo(Tyr-Ala) (FIGS. 10a and 10b) display the same RP-HPLC retention times and fragmentation patterns than that of the metabolites detected in the culture supernatant of pEXP-Rv2275.

Definitely, we showed that expression of Rv2275 in E. coli leads to the synthesis of cyclo(Tyr-Tyr), cyclo(Tyr-Ala), cyclo(Tyr-Phe) and cyclo(Tyr-Trp) found in the culture medium, demonstrating that Rv2275 is a cyclo(Tyr-Xaa)-synthesizing enzyme that can be produced in an active form in E. coli.

### EXAMPLE 4

### Construction of an Escherichia coli Expression Vector Encoding a Truncated Rv2275 Protein (SEQ ID NO: 3) as a C-Terminal His₆-Tagged Fusion

Rv2275 is 289 amino acids long, compared to 239 for AlbC. The alignment of both proteins shows that in Rv2275 there is an N-terminal extension of 48 amino acids, which has no equivalent in AlbC. To address the question of the dispensability of several amino acids, two constructions were made to express two different C-terminal His₆-tagged versions of Rv2275 in the cloning vector pQE60 (Qiagen).

In the first construction, named plasmid pQE60-Rv2275L, the complete coding sequence of Rv2275 is present and in the second construction, named plasmid pQE60-Rv2275C, a truncated coding sequence lacking the first 48 codons is present.

Construction of the plasmid pQE60-Rv2275L

A DNA fragment carrying the complete sequence encoding Rv2275 was obtained after PCR amplification using pEXP-Rv2275 as template, Taq DNA polymerase (Pharmacia) and the following primers under conditions recommended by the manufacturer:

primer KRVLF
(SEQ ID NO: 15)
(5′-CGGCCATGGCATACGTGGCTGCCGAACCAGGC-3′,
NcoI site underlined),
and
primer KRVR
(SEQ ID NO: 16)
(5′-GGCAGATCTTTCGGCGGGGCTCCCATCAGG-3′,
BglII site underlined).

Two restriction sites were thus introduced: a NcoI site upstream of the coding sequence and a BglII site downstream. The introduction of the NcoI site was accompanied by the replacement of the second codon TCA by GCA (see SEQ ID NO: 5 wherein K is G), leading to the replacement of the second amino acid serine by alanine in the corresponding protein (see SEQ ID NO: 6 wherein the amino acid at location 2 stands for Ala). This DNA fragment was cloned as a NcoI-BglII fragment into the vector pQE60 digested by NcoI and BamHI, yielding the plasmid pQE60-Rv2275L (SEQ ID NO: 20). The DNA sequence of the insert was confirmed by sequencing.

Construction of the Plasmid pQE60-Rv2275C

A DNA fragment carrying the sequence encoding the truncated version of Rv2275 was obtained after PCR amplification using pEXP-Rv2275 as template, Taq DNA polymerase (Pharmacia) and the following primers under conditions recommended by the manufacturer:

primer KRVCF
(SEQ ID NO: 17)
(5′-CGGCCATGGAGCTAGGCAGGCGCATTCCGGAAGC-3′,
NcoI site underlined),
and
primer KRVR
(SEQ ID NO: 16)
(5′-GGCAGATCTTTCGGCGGGGCTCCCATCAGG-3′,
BglII site underlined).

Two restriction sites were thus introduced: a NcoI site upstream of the coding sequence and a BglII site downstream. In the fragment obtained by PCR amplification an ATG initiation codon was introduced at a position corresponding to the 49^{th} codon (TTT) in the original Rv2275 sequence. The introduction of the NcoI site was also accompanied by the replacement of the codon CAG (50^{th} codon in the original sequence) by GAG (second codon in the truncated sequence) (see SEQ ID NO: 1 wherein S is G), leading to the replacement of a glutamine by glutamic acid in the corresponding protein (see SEQ ID NO: 3 wherein the amino acid at location stands for Glu). This DNA fragment was cloned as a NcoI-BglII fragment into the vector pQE60 digested by NcoI and BamHI, yielding the plasmid pQE60-Rv2275C (SEQ ID NO: 21). The DNA sequence of the insert was confirmed by sequencing.

### Expression of the Complete and Truncated Rv2275 Protein for Synthesis of Cyclodipeptides

The expressions of the complete and truncated versions of Rv2275 were performed by cultivating E. coli cells harboring the plasmid pQE60-Rv2275L or pQE60-Rv2275C, as described in Example 2.

Expressions of C-terminal tagged Rv2275 from plasmids pQE60-Rv2275L and pQE60-Rv2275C were made by using E. coli strain M15 containing the pREP4 plasmid (hereinafter referred as to “M15pREP4”) (Qiagen). 50 μl chemically competent cells were transformed with 20 ng plasmid pQE60-Rv2275L, pQE60-Rv2275C and pQE60 using standard heat-shock procedure (Sambrook et al., aforementioned). After 1 h outgrowth in SOC medium, transformation mixture was spread on LB plates containing 0.5% glucose, 100 μg/ml ampicillin and 30 μg/ml kanamycin and incubated overnight at 37° C. A few colonies were picked up to inoculate M9 liquid medium supplemented with vitamins and oligoelements solutions containing 0.5% glucose, 100 μg/ml ampicillin and 30 μg/ml kanamycin. After 24 h growth at 37° C. with 200 rpm on a rotary shaker, 25 ml preheated minimal medium supplemented with vitamins and oligoelements solutions containing 0.5% glycerol, 100 μg/ml ampicillin and 30 μg/ml kanamycin were inoculated with 500 μl starter culture in minimal medium. Bacterial cultures were incubated at 37° C. with 200 rpm rotary shaking and absorbance at 600 nm (OD₆₀₀) was followed during the growth. When OD₆₀₀ reached 0.5, 1 mM IPTG was added and cultures were incubated with 200 rpm rotary shaking at 20° C. for 20 hours. Bacterial cultures were then centrifuged 20 min. at 3,000 g and supernatant was saved for cyclodipeptide production analysis.

E. coli M15pREP4 cells harbouring the plasmid pQE60-Rv2275L were cultivated and their ability to synthesize cyclo(Tyr-Tyr), cyclo(Tyr-Ala), cyclo(Tyr-Phe) and cyclo(Tyr-Trp) was evaluated by HPLC as previously described. The chromatogram at 220 nm was found similar to that obtained with supernatant of E. coli cells harbouring the plasmid pEXP-Rv2275 (data not shown).

E. coli M15pREP4 cells harbouring the plasmid pQE60-Rv2275C were cultivated and their ability to synthesize cyclo(Tyr-Tyr), cyclo(Tyr-Ala), cyclo(Tyr-Phe) and cyclo(Tyr-Trp) was evaluated by HPLC as previously described (FIG. 11). The chromatogram at 220 nm was found similar to that obtained with supernatant of E. coli cells harbouring the plasmid pEXP-Rv2275 (compare chromatograms in FIG. 3 and FIG. 11): expression of the truncated version of Rv2275 led to the presence of four peaks (quoted peak 1, 2, 3 and 4 in 11) that display respectively retention times and spectral characteristics similar to those of peaks 1, 2, 3 and 4 previously obtained with full length Rv2275 (FIG. 3). As revealed by MS and MSMS analysis (FIGS. 12 & 13), peaks 1′, 2′, 3′ and 4′, were respectively identified as cyclo(Tyr-Ala), cyclo(Tyr-Tyr), cyclo(Tyr-Trp) and cyclo(Tyr-Phe). Therefore the N-terminal extension of Rv2275 is dispensable for the cyclo(Tyr-Xaa) synthesizing activity.

### EXAMPLE 5

### In Vitro Production of Cyclo(Tyr-Tyr) by the Purified Rv2275 Protein

Production of the Purified Rv2275 Protein

Bacterial culture for production of the Rv2275 protein was performed as already described in Example 2, except that minimal medium was replaced by LB medium (Sambrook et al., aforementioned). After induction with 0.02% arabinose, the culture was continued at 20° C. for 12 h. The bacterial cells were harvested by centrifugation at 4,000 g for 20 min and frozen at −80° C. Then, bacterial cells were thawed and resuspended in 1.5 ml of an extraction buffer composed of 100 mM Tris-HCl pH 8, 150 mM NaCl and 5% glycerol. Cells were broken using an Eaton press and centrifuged at 20,000 g and 4° C. for 20 min. The resulting supernatant containing the soluble proteins was loaded onto a Ni²⁺-column (HisTrap HP from Amersham) equilibrated with a buffer composed of 100 mM Tris-HCl pH 8, 150 mM NaCl. The column was washed with the same buffer and submitted to a linear gradient of imidazole (from 0 to 1 M imidazole at pH 8). The Rv2275 protein was eluted at around 250 mM imidazole. The purified Rv2275 protein was then washed (to eliminate imidazole) and concentrated using a Vivaspin concentrator (Vivascience).

Preparation of the Soluble Cell Extract Used for Supplementation

Bacteria transformed with the empty vector pQE60 (Qiagen) were cultivated and broken as previously described. The broken cells were centrifuged at 20,000 g and 4° C. for 20 min. The resulting supernatant corresponds to a soluble extract of E. coli cells, which does not contain cyclodipeptide synthetase.

In Vitro Production of Cyclo(Tyr-Tyr)

A 215 μl-reaction mixture comprising 5.5 mM Tyr, 10 mM ATP, 20 mM MgCl₂ and 25 μM of the purified Rv2275 protein was supplemented with 115 μl of the previously described soluble cell extract. This mixture was incubated at 30° C. for 12 h. The reaction was stopped by adding TFA and submitted to a centrifugation at 20,000 g for 20 min. The supernatant was then analyzed by HPLC and HPLC-eluted fractions were characterized by mass spectrometry as described in Example 2. As a control, the same experiment was performed under similar conditions except that the purified Rv2275 was omitted.

The results clearly showed that the incubated mixture comprising the Rv2275 protein contains cyclo(Tyr-Tyr) (an HPLC-eluted fraction at a retention time of 21.3 min with mass characteristics similar to that shown in FIG. 5) whereas the incubated mixture devoid of the Rv2275 protein contains no cyclodipeptide. This demonstrates that the formation of cyclo(Tyr-Tyr) can be performed in vitro with a purified cyclo(Tyr-Xaa) synthetase.

The procedure described for the cyclo(Tyr-Tyr) cyclodipeptide can be applied to cyclo(Tyr-Phe), cyclo(Tyr-Trp) and cyclo(Tyr-Ala).

1-22. (canceled)

23. A method for the synthesis of cyclo(Tyr-Xaa) cyclodipeptides, wherein Xaa is any amino acid, which comprises the steps of:
(1) incubating two amino acids Tyr and Xaa, which are identical or different, under suitable conditions, with an isolated cyclodipeptide synthetase, which:
a) has an ability to produce cyclo(Tyr-Xaa) cyclodipeptides from two amino acids Tyr and Xaa, wherein Xaa is any amino acid, and
b) comprises a polypeptide sequence having at least 40% identity or at least 60% similarity with the polypeptide of the sequence SEQ ID NO: 3; and
(2) recovering the cyclo(Tyr-Xaa) cyclodipeptides thus obtained.

24. A method for the synthesis of α,β-dehydrogenated cyclo(Tyr-Xaa) cyclodipeptides, wherein Xaa is any amino acid, which comprises the steps of:
(1) incubating two amino acids Tyr and Xaa, which may be identical or different under suitable conditions with an isolated cyclodipeptide synthetase, which:
a) has an ability to produce cyclo(Tyr-Xaa) cyclodipeptides from two amino acids Tyr and Xaa, wherein Xaa is any amino acid, and
b) comprises a polypeptide sequence having at least 40% identity or at least 60% similarity with the polypeptide of the sequence SEQ ID NO: 3;
and a purified CDO, and
(2) recovering the α,β-dehydrogenated cyclodipeptides.

25. The method according to claim 23, wherein step (1) is performed in a presence of suitable amino acids at a concentration between 0.1 mM to 100 mM, a cyclodipeptide synthetase at a concentration between 0.1 nM and 100 μM, in a buffer at a pH between 6 and 8, and containing a soluble extract of prokaryote cells such as E. coli or Streptomyces cells which do not produce cyclodipeptide synthetase.

26. The method according to claim 23, which further comprises a preliminary step wherein a polynucleotide which is selected from the group consisting of:
a) a polynucleotide encoding an isolated cyclodipeptide synthetase, which:
i) has an ability to produce cyclo(Tyr-Xaa) cyclodipeptides from two amino acids Tyr and Xaa, wherein Xaa is any amino acid, and
ii) comprises a polypeptide sequence having at least 40% identity or at least 60% similarity with the polypeptide of the sequence SEQ ID NO: 3;
b) a complementary polynucleotide of the polynucleotide a), and
c) a polynucleotide which hybridizes to polynucleotide a) or b) under stringent hybridization conditions;
is used for synthesizing the cyclodipeptide synthetase, which is performed before step (1).

27. A method for the synthesis of cyclo(Tyr-Xaa) cyclodipeptides, wherein Xaa is any amino acid, comprising the following steps:
(1) culturing a host cell modified by an isolated polynucleotide, which is selected from the group consisting of:
a) a polynucleotide encoding an isolated cyclodipeptide synthetase, which:
i) has an ability to produce cyclo(Tyr-Xaa) cyclodipeptides from two amino acids Tyr and Xaa, wherein Xaa is any amino acid, and
ii) comprises a polypeptide sequence having at least 40% identity or at least 60% similarity with the polypeptide of the sequence SEQ ID NO: 3;
b) a complementary polynucleotide of the polynucleotide a), and
c) a polynucleotide which hybridizes to polynucleotide a) or b) under stringent hybridization conditions;
in suitable culture conditions for said host, and
(2) recovering the cyclodipeptides from the culture medium.

28. A method for the synthesis of α,β-dehydrogenated cyclo(Tyr-Xaa) cyclodipeptides, wherein Xaa is any amino acid, comprising the following steps:
(1) culturing a host cell modified by an isolated polynucleotide, which is selected from the group consisting of:
a) a polynucleotide encoding an isolated cyclodipeptide synthetase, which:
i) has an ability to produce cyclo(Tyr-Xaa) cyclodipeptides from two amino acids Tyr and Xaa, wherein Xaa is any amino acid, and
ii) comprises a polypeptide sequence having at least 40% identity or at least 60% similarity with the polypeptide of the sequence SEQ ID NO: 3;
b) a complementary polynucleotide of the polynucleotide a), and
c) a polynucleotide which hybridizes to polynucleotide a) or b) under stringent hybridization conditions;
in suitable culture conditions for said host,
(2) incubating the cyclo(Tyr-Xaa) cyclodipeptide obtained from step (1) with a purified CDO, and
(3) recovering the cyclodipeptides from the culture medium.

29. The method according to claim 25, wherein said suitable amino acids are in a concentration between 1 mM to 10 mM.

30. The method according to claim 25, wherein said cyclopeptide synthetase is in a concentration between 1 μM to 100 μM.

31. The method according to claim 24, wherein step (1) is performed in presence of suitable amino acids at a concentration between 0.1 mM to 100 mM, a cyclodipeptide synthetase at a concentration between 0.1 nM and 100 μM, in a buffer at a pH between 6 and 8, and containing a soluble extract of prokaryote cells such as E. coli or Streptomyces cells which do not produce cyclodipeptide synthetase.

32. The method according to claim 24, which further comprises a preliminary step wherein a polynucleotide which is selected from the group consisting of:
a) a polynucleotide encoding an isolated cyclodipeptide synthetase, which:
i) has an ability to produce cyclo(Tyr-Xaa) cyclodipeptides from two amino acids Tyr and Xaa, wherein Xaa is any amino acid, and
ii) comprises a polypeptide sequence having at least 40% identity or at least 60% similarity with the polypeptide of the sequence SEQ ID NO: 3;
b) a complementary polynucleotide of the polynucleotide a), and
c) a polynucleotide which hybridizes to polynucleotide a) or b) under stringent hybridization conditions;
is used for synthesizing the cyclodipeptide synthetase, which is performed before step (1).


SEQUENCE LISTING (22 sequences)

SEQ ID NO: 1 - DNA, 726 bp, Mycobacterium tuberculosis
atgsagctaggcaggcgcattccggaagccaccgcccaggaagggtttctggttcggccattcacccaacaatgtcagatcatccacaccgaaggagatcatgctgttatcggggtatccccggggaacagttacttctcccgccagcgcctacgggatctcgggctttggggtctcacgaattttgatcgtgtggacttcgtctacaccgatgtccatgtcgccgagagttacgaagcgctaggcgattccgcaatcgaagcccggcgcaaggcggtcaaaaacatccgcggcgtccgcgccaagatcaccaccacggtgaacgaactcgatccggccggggcccggctgtgcgttcgtccgatgtcggagttccagtccaacgaggcataccgggagctgcatgcggacctgctcacgcgcctgaaagacgacgaggacttgcgcgccgtctgccaggacctagtgcggcgcttcctgtccacgaaagtgggtccgcggcagggggcgacggctactcaagagcaggtgtgcatggactacatttgcgccgaggccccgctattcctcgacacacctgcgattctcggagtgccgtcgtcgttgaattgctaccaccaatcactgcccctcgccgaaatgctctacgcccgaggatcgggactacgggcatcgcgcaatcaaggccacgccattgttacccctgatgggagccccgccgaatga

SEQ ID NO: 2 - DNA, 870 bp, Mycobacterium tuberculosis
atgtcatacgtggctgccgaaccaggcgtgctgatctcgccgacggacgacttgcagagcccccggtcagccccggcagcgcatgacgaaaatgcggacggcataacaggcgggaccagagacgactctgctcccaactcacggtttcagctaggcaggcgcattccggaagccaccgcccaggaagggtttctggttcggccattcacccaacaatgtcagatcatccacaccgaaggagatcatgctgttatcggggtatccccggggaacagttacttctcccgccagcgcctacgggatctcgggctttggggtctcacgaattttgatcgtgtggacttcgtctacaccgatgtccatgtcgccgagagttacgaagcgctaggcgattccgcaatcgaagcccggcgcaaggcggtcaaaaacatccgcggcgtccgcgccaagatcaccaccacggtgaacgaactcgatccggccggggcccggctgtgcgttcgtccgatgtcggagttccagtccaacgaggcataccgggagctgcatgcggacctgctcacgcgcctgaaagacgacgaggacttgcgcgccgtctgccaggacctagtgcggcgcttcctgtccacgaaagtgggtccgcggcagggggcgacggctactcaagagcaggtgtgcatggactacatttgcgccgaggccccgctattcctcgacacacctgcgattctcggagtgccgtcgtcgttgaattgctaccaccaatcactgcccctcgccgaaatgctctacgcccgaggatcgggactacgggcatcgcgcaatcaaggccacgccattgttacccctgatgggagccccgccgaatga

SEQ ID NO: 3 - protein, 241 aa, Mycobacterium tuberculosis
  MOD_RES (2)..(2): Gln or Glu
MXLGRRIPEATAQEGFLVRPFTQQCQIIHTEGDHAVIGVSPGNSYFSRQRLRDLGLWGLTNFDRVDFVYTDVHVAESYEALGDSAIEARRKAVKNIRGVRAKITTTVNELDPAGARLCVRPMSEFQSNEAYRELHADLLTRLKDDEDLRAVCQDLVRRFLSTKVGPRQGATATQEQVCMDYICAEAPLFLDTPAILGVPSSLNCYHQSLPLAEMLYARGSGLRASRNQGHAIVTPDGSPAE

SEQ ID NO: 4 - protein, 289 aa, Mycobacterium tuberculosis
MSYVAAEPGVLISPTDDLQSPRSAPAAHDENADGITGGTRDDSAPNSRFQLGRRIPEATAQEGFLVRPFTQQCQIIHTEGDHAVIGVSPGNSYFSRQRLRDLGLWGLTNFDRVDFVYTDVHVAESYEALGDSAIEARRKAVKNIRGVRAKITTTVNELDPAGARLCVRPMSEFQSNEAYRELHADLLTRLKDDEDLRAVCQDLVRRFLSTKVGPRQGATATQEQVCMDYICAEAPLFLDTPAILGVPSSLNCYHQSLPLAEMLYARGSGLRASRNQGHAIVTPDGSPAE

SEQ ID NO: 5 - DNA, 870 bp, Mycobacterium tuberculosis
  CDS (1)..(870)
atggcatacgtggctgccgaaccaggcgtgctgatctcgccgacggacgacttgcagagcccccggtcagccccggcagcgcatgacgaaaatgcggacggcataacaggcgggaccagagacgactctgctcccaactcacggtttcagctaggcaggcgcattccggaagccaccgcccaggaagggtttctggttcggccattcacccaacaatgtcagatcatccacaccgaaggagatcatgctgttatcggggtatccccggggaacagttacttctcccgccagcgcctacgggatctcgggctttggggtctcacgaattttgatcgtgtggacttcgtctacaccgatgtccatgtcgccgagagttacgaagcgctaggcgattccgcaatcgaagcccggcgcaaggcggtcaaaaacatccgcggcgtccgcgccaagatcaccaccacggtgaacgaactcgatccggccggggcccggctgtgcgttcgtccgatgtcggagttccagtccaacgaggcataccgggagctgcatgcggacctgctcacgcgcctgaaagacgacgaggacttgcgcgccgtctgccaggacctagtgcggcgcttcctgtccacgaaagtgggtccgcggcagggggcgacggctactcaagagcaggtgtgcatggactacatttgcgccgaggccccgctattcctcgacacacctgcgattctcggagtgccgtcgtcgttgaattgctaccaccaatcactgcccctcgccgaaatgctctacgcccgaggatcgggactacgggcatcgcgcaatcaaggccacgccattgttacccctgatgggagccccgccgaatga

SEQ ID NO: 6 - protein, 289 aa, Mycobacterium tuberculosis
MAYVAAEPGVLISPTDDLQSPRSAPAAHDENADGITGGTRDDSAPNSRFQLGRRIPEATAQEGFLVRPFTQQCQIIHTEGDHAVIGVSPGNSYFSRQRLRDLGLWGLTNFDRVDFVYTDVHVAESYEALGDSAIEARRKAVKNIRGVRAKITTTVNELDPAGARLCVRPMSEFQSNEAYRELHADLLTRLKDDEDLRAVCQDLVRRFLSTKVGPRQGATATQEQVCMDYICAEAPLFLDTPAILGVPSSLNCYHQSLPLAEMLYARGSGLRASRNQGHAIVTPDGSPAE

SEQ ID NO: 7 - DNA, 35 bp, Artificial Sequence
  Description of Artificial Sequence Synthetic primer
ccgtccctatggtccaaggaaaacaatgtcatacg

SEQ ID NO: 8 - DNA, 34 bp, Artificial Sequence
  Description of Artificial Sequence Synthetic primer
gcaagcaataacggcggggctcccatcaggggta

SEQ ID NO: 9 - DNA, 42 bp, Artificial Sequence
  Description of Artificial Sequence Synthetic primer
ggcttcgagaatctttattttcagggctcatacgtggctgcc

SEQ ID NO: 10 - DNA, 47 bp, Artificial Sequence
  Description of Artificial Sequence Synthetic primer
ggggaccactttgtacaagaaagctgggtccttattcggcggggctc

SEQ ID NO: 11 - DNA, 47 bp, Artificial Sequence
  Description of Artificial Sequence Synthetic primer
ggggacaagtttgtacaaaaaagcaggcttcgagaatctttattttc

SEQ ID NO: 12 - DNA, 20 bp, Artificial Sequence
  Description of Artificial Sequence Synthetic primer
tcggccattcacccaacaat

SEQ ID NO: 13 - DNA, 15 bp, Artificial Sequence
  Description of Artificial Sequence Synthetic primer
gtaaacgacggccag

SEQ ID NO: 14 - DNA, 17 bp, Artificial Sequence
  Description of Artificial Sequence Synthetic primer
caggaaacagctatgac

SEQ ID NO: 15 - DNA, 32 bp, Artificial Sequence
  Description of Artificial Sequence Synthetic primer
cggccatggcatacgtggctgccgaaccaggc

SEQ ID NO: 16 - DNA, 30 bp, Artificial Sequence
  Description of Artificial Sequence Synthetic primer
ggcagatctttcggcggggctcccatcagg

SEQ ID NO: 17 - DNA, 34 bp, Artificial Sequence
  Description of Artificial Sequence Synthetic primer
cggccatggagctaggcaggcgcattccggaagc

SEQ ID NO: 18 - protein, 29 aa, Artificial Sequence
  Description of Artificial Sequence Synthetic peptide
MSYYHHHHHHLESTSLYKKAGFENLYFQG

SEQ ID NO: 19 - DNA, 5591 bp, Artificial Sequence
  Description of Artificial Sequence Synthetic polynucleotide
agatctcgatcccgcgaaattaatacgactcactatagggagaccacaacggtttccctctagaaataattttgtttaactttaagaaggagatatacatatgtcgtactaccatcaccatcaccatcacctcgaatcaacaagtttgtacaaaaaagcaggcttcgagaatctttattttcagggctcatacgtggctgccgaaccaggcgtgctgatctcgccgacggacgacttgcagagcccccggtcagccccggcagcgcatgacgaaaatgcggacggcataacaggcgggaccagagacgactctgctcccaactcacggtttcagctaggcaggcgcattccggaagccaccgcccaggaagggtttctggttcggccattcacccaacaatgtcagatcatccacaccgaaggagatcatgctgttatcggggtatccccggggaacagttacttctcccgccagcgcctacgggatctcgggctttggggtctcacgaattttgatcgtgtggacttcgtctacaccgatgtccatgtcgccgagagttacgaagcgctaggcgattccgcaatcgaagcccggcgcaaggcggtcaaaaacatccgcggcgtccgcgccaagatcaccaccacggtgaacgaactcgatccggccggggcccggctgtgcgttcgtccgatgtcggagttccagtccaacgaggcataccgggagctgcatgcggacctgctcacgcgcctgaaagacgacgaggacttgcgcgccgtctgccaggacctagtgcggcgcttcctgtccacgaaagtgggtccgcggcagggggcgacggctactcaagagcaggtgtgcatggactacatttgcgccgaggccccgctattcctcgacacacctgcgattctcggagtgccgtcgtcgttgaattgctaccaccaatcactgcccctcgccgaaatgctctacgcccgaggatcgggactacgggcatcgcgcaatcaaggccacgccattgttacccctgatgggagccccgccgaataaggacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 20 - DNA, 4286 bp, Artificial Sequence
  Description of Artificial Sequence Synthetic polynucleotide
ctcgagaaatcataaaaaatttatttgctttgtgagcggataacaattataatagattcaattgtgagcggataacaatttcacacagaattcattaaagaggagaaattaaccatggcatacgtggctgccgaaccaggcgtgctgatctcgccgacggacgacttgcagagcccccggtcagccccggcagcgcatgacgaaaatgcggacggcataacaggcgggaccagagacgactctgctcccaactcacggtttcagctaggcaggcgcattccggaagccaccgcccaggaagggtttctggttcggccattcacccaacaatgtcagatcatccacaccgaaggagatcatgctgttatcggggtatccccggggaacagttacttctcccgccagcgcctacgggatctcgggctttggggtctcacgaattttgatcgtgtggacttcgtctacaccgatgtccatgtcgccgagagttacgaagcgctaggcgattccgcaatcgaagcccggcgcaaggcggtcaaaaacatccgcggcgtccgcgccaagatcaccaccacggtgaacgaactcgatccggccggggcccggctgtgcgttcgtccgatgtcggagttccagtccaacgaggcataccgggagctgcatgcggacctgctcacgcgcctgaaagacgacgaggacttgcgcgccgtctgccaggacctagtgcggcgcttcctgtccacgaaagtgggtccgcggcagggggcgacggctactcaagagcaggtgtgcatggactacatttgcgccgaggccccgctattcctcgacacacctgcgattctcggagtgccgtcgtcgttgaattgctaccaccaatcactgcccctcgccgaaatgctctacgcccgaggatcgggactacgggcatcgcgcaatcaaggccacgccattgttacccctgatgggagccccgccgaaagatctcatcaccatcaccatcactaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 21 - DNA, 4142 bp, Artificial Sequence
  Description of Artificial Sequence Synthetic polynucleotide
ctcgagaaatcataaaaaatttatttgctttgtgagcggataacaattataatagattcaattgtgagcggataacaatttcacacagaattcattaaagaggagaaattaaccatggagctaggcaggcgcattccggaagccaccgcccaggaagggtttctggttcggccattcacccaacaatgtcagatcatccacaccgaaggagatcatgctgttatcggggtatccccggggaacagttacttctcccgccagcgcctacgggatctcgggctttggggtctcacgaattttgatcgtgtggacttcgtctacaccgatgtccatgtcgccgagagttacgaagcgctaggcgattccgcaatcgaagcccggcgcaaggcggtcaaaaacatccgcggcgtccgcgccaagatcaccaccacggtgaacgaactcgatccggccggggcccggctgtgcgttcgtccgatgtcggagttccagtccaacgaggcataccgggagctgcatgcggacctgctcacgcgcctgaaagacgacgaggacttgcgcgccgtctgccaggacctagtgcggcgcttcctgtccacgaaagtgggtccgcggcagggggcgacggctactcaagagcaggtgtgcatggactacatttgcgccgaggccccgctattcctcgacacacctgcgattctcggagtgccgtcgtcgttgaattgctaccaccaatcactgcccctcgccgaaatgctctacgcccgaggatcgggactacgggcatcgcgcaatcaaggccacgccattgttacccctgatgggagccccgccgaaagatctcatcaccatcaccatcactaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 22 - protein, 6 aa, Artificial Sequence
  Description of Artificial Sequence Synthetic 6x His tag
HHHHHH